(19) United States
(12) Patent Application Publication
(10) Pub. No.: US 2012/0330283 A1
(43) Pub. Date: Dec. 27, 2012

(54) Systems, devices, and methods to induce programmed cell death in adipose tissue

(75) Inventors: Roderick A. Hyde, Redmond, WA (US); Muriel Y. Ishikawa, Livermore, CA (US); Jordin T. Kare, Seattle, WA (US); Eric C. Leuthardt, St. Louis, MO (US); Dennis J. Rivet, Chesapeake, VA (US); Michael A. Smith, Phoenix, AZ (US); Elizabeth A. Sweeney, Seattle, WA (US); Lowell L. Wood, JR., Bellevue, WA (US)

(73) Assignee: Elwha LLC, a limited liability company of the State of Delaware

(21) Appl. No.: 13/135,127

(22) Filed: Jun. 23, 2011

Related U.S. Application Data
(63) Continuation of application No. 13/135,131, filed on Jun. 23, 2011.

(51) Int. Cl. A61B 17/00

(52) U.S. Cl. 606/1

(57) ABSTRACT

Systems, devices, and methods are described for registering treatment focal regions with adipose depot targets and for transcutaneously delivering an energy stimulus to the treatment focal regions when treatment registration information indicates that the treatment focal regions coincide with the anatomical targets.

### SUMMARY

In an aspect, the present disclosure is directed to, among other things, an energy delivery apparatus including a target registration module configured to align at least one treatment focal region with one or more adipose depot targets. In an embodiment the delivery apparatus includes an apoptosis inducement module configured to deliver a pro-apoptotic energy stimulus to one or more treatment focal regions according to a treatment cycle based on an induced apoptosis to necrosis comparison.

In an aspect, the present disclosure is directed to, among other things, a system including a target registration module configured to align a plurality of treatment focal regions with one or more adipose depot targets. In an embodiment, the system includes an apoptosis inducement module configured to deliver a pro-apoptotic energy stimulus to the plurality of treatment focal regions. In an embodiment, the apoptosis inducement module alters a duty cycle associated with a delivery of the pro-apoptotic energy stimulus to the plurality of treatment focal regions based on at least one measurand associated with the one or more adipose depot targets.

In an aspect, the present disclosure is directed to, among other things, a transcutaneous energy delivery apparatus including a target identification and registration module configured to identify a treatment target of a biological subject (e.g., a patient, user, etc.) based on a detected measurand, and to align a plurality of treatment focal regions with the treatment target. In an embodiment, the transcutaneous energy delivery apparatus includes an apoptosis inducement module configured to determine a treatment protocol of pro-apoptotic energy and to transcutaneously deliver pro-apoptotic energy to the at least one treatment target according to the treatment protocol.

In an aspect, the present disclosure is directed to, among other things, a multi-pass transcutaneous energy delivery method including registering a first plurality of anatomical targets to reference treatment registration data. In an embodiment, the multi-pass transcutaneous energy delivery method includes transcutaneously delivering a pro-apoptotic energy stimulus to the first plurality of anatomical targets. In an embodiment, the multi-pass transcutaneous energy delivery method includes registering a second plurality of anatomical targets to reference treatment registration data. In an embodiment, the multi-pass transcutaneous energy delivery method includes transcutaneously delivering a pro-apoptotic energy stimulus to the second plurality of anatomical targets.

In an aspect, the present disclosure is directed to, among other things, a multi-pass transcutaneous energy delivery method including registering at least one treatment focal region within a biological subject with at least one adipocyte target. In an embodiment, the multi-pass transcutaneous energy delivery method includes determining whether the adipocyte target has been treated. In an embodiment, the multi-pass transcutaneous energy delivery method includes transcutaneously delivering a pro-apoptotic energy stimulus to the adipocyte target based on the determination.

In an aspect, the present disclosure is directed to, among other things, a method including generating first-in-time treatment registration information indicative of an alignment of at least one treatment focal region with a first adipose depot target. In an embodiment, the method includes transcutaneously delivering a pro-apoptotic energy stimulus to the at least one treatment focal region based on the first-in-time treatment registration information.

In an aspect, the present disclosure is directed to, among other things, a transcutaneous energy delivery apparatus, including a target registration means for aligning a treatment focal region with an adipose depot target and for generating treatment protocol information. In an embodiment, the transcutaneous energy delivery apparatus includes an apoptosis induction means for transcutaneously delivering an energy stimulus to the at least one treatment focal region. In an embodiment, the transcutaneous energy delivery apparatus includes a target tracking means including a sensor component and a computing device operably coupled to the sensor component and the apoptosis induction means. In an embodiment, the target tracking means registers a treatment focal region location within the body of a biological subject relative to a reference location and alters a duty cycle associated with the transcutaneous delivery of the energy stimulus based on the registering of the treatment focal region location within the body relative to the reference location.

In an aspect, the present disclosure is directed to, among other things, a transcutaneous energy delivery apparatus including a real-time registration module configured to registered one or more treatment focal regions with at least one anatomical target and to generate treatment registration information. In an embodiment, the transcutaneous energy delivery apparatus includes an apoptosis induction module configured to transcutaneously deliver a pro-apoptotic energy stimulus to the one or more treatment focal regions based on the treatment registration information.

### BRIEF DESCRIPTION OF THE FIGURES

FIG. 1 is a perspective view of a system including a target registration module and an apoptosis inducement module according to one embodiment.

FIG. 2 is a perspective view of a transcutaneous energy delivery apparatus according to one embodiment.

FIG. 3 is a perspective view of an energy delivery apparatus according to one embodiment.

FIGS. 4A, 4B, 4C, 4D and 4E show a flow diagram of a method according to one embodiment.

FIGS. 5A and 5B show a flow diagram of a according to one embodiment.

FIG. 6 shows a flow diagram of a method according to one embodiment.

FIG. 7 shows a flow diagram of a method according to one embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

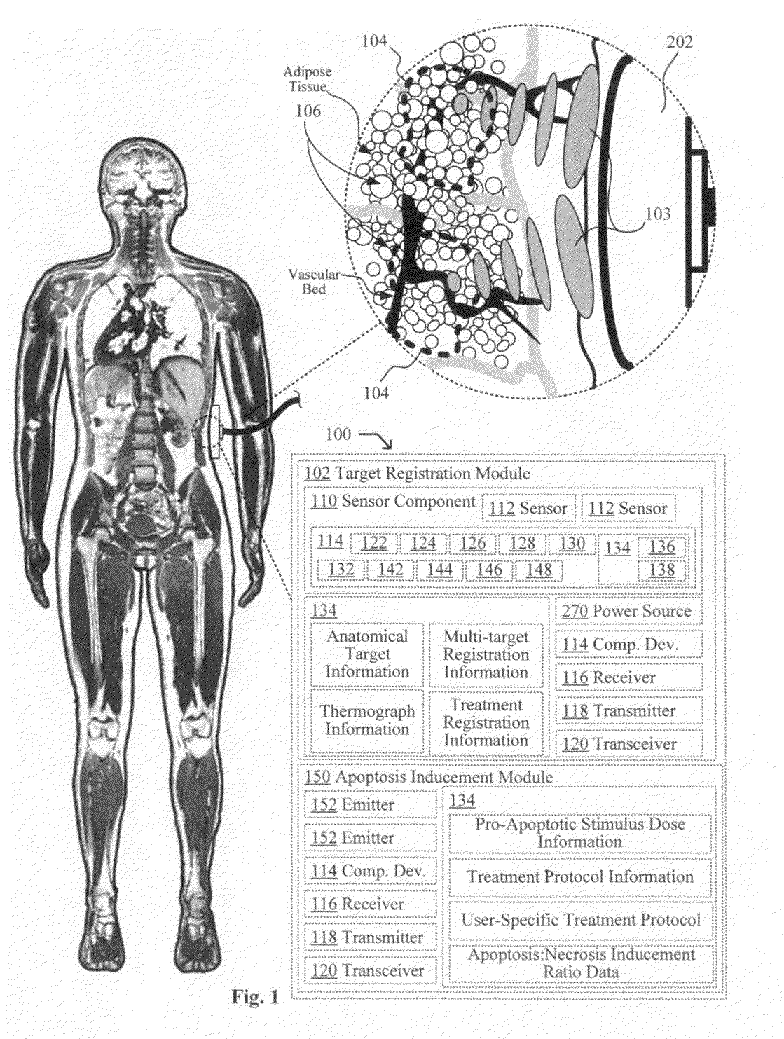

FIGS. 1, 2, and 3 shows systems 100 (e.g., a medical treatment system, an energy delivery system, a transcutaneous energy delivery system, a multi-pass transcutaneous energy delivery system, or the like) devices (e.g., energy delivery apparatuses , transcutaneous energy delivery apparatuses 202, etc.), etc., in which one or more methodologies or technologies can be implemented such as, for example, inducing programmed cell death (PCD), without substantially inducing necrosis, of adipocytes within one or more treatment focal regions, or the like.

In an embodiment, the system 100 includes a target registration module 102 configured to register a plurality of treatment focal regions 104 with one or more adipose depot targets 106. In an embodiment, the target registration module 102 registers a treatment target (e.g., an adipose depot target, or the like) with a treatment focal region (e.g., a focal area, a focal zone, a focal volume, energy stimulus focal region, energy concentration region, energy convergence region, or the like) using one or more registration techniques or methodologies. For example, during the delivery of a pro-apoptotic energy stimulus 103, the target registration module 102 maps (e.g., spatially aligns, etc.) a treatment focal region to a treatment target. In an embodiment, the target registration module 102 registers a plurality of objects by mapping coordinates from one object to corresponding points in.another object. In an embodiment, the target registration module 102 registers objects (e.g., target and reference objects, treatment targets and treatment focal regions, images, etc.) using transformations.

Non-limiting examples of registration techniques or methodologies include deformable registration, landmark-based registration, or rigid registration. See e.g., Paquin et al., Multiscale Image Registration, Mathematical Biosciences and Engineering, Vol. 3:2 (2006); see also Paquin, Dana, PhD, Multiscale Methods for Image Registration, Ph.D. dissertation, Stanford University (2007); Zitova et al., Image Registration Methods: a Survey, Image and Vision Computing (21) pp. 977-1000 (2003); each of which is incorporated herein by reference. In an embodiment, registration includes techniques or methodologies for spatially aligning images taken using different imaging modalities, taken at different times, or that vary in perspective. Further non-limiting examples of registration techniques or methodologies include deformable multiscale registration, hybrid multiscale landmark registration, multiscale image registration, or rigid multiscale registration. In an embodiment, registration includes one or more of feature detection, feature identification, feature matching, or transform modeling. In an embodiment, registration includes mapping features of a first object with the features of a second object. In an embodiment, registration includes determining a point-by-point correspondence between two objects (e.g., a treatment focal region and a treatment target, etc.).

In an embodiment, the target registration module 102 generates a three dimensional reconstruction of an anatomical feature or an internal structure using one or more registration techniques or methodologies. For example, in an embodiment, the target registration module 102 generates a three dimensional reconstruction of a vascular structure registering a plurality of infrared images. Further non-limiting examples of registration techniques or methodologies include feature-based registration, fiducial-based registration, landmark-based registration, non-parametric image registration, optimal parametric registration, principal-axes-based registration, scan-based registration, or surface-based registration. Further non-limiting examples of registration techniques or methodologies include atlas based registration methods, correlation based registration methods, curve matching based registration methods, moment and principal axes based registration methods, mutual information based registration methods, surface matching based registration methods, or wavelet based registration methods.

In an embodiment, the target registration module 102 registers images taken from a variety of sensors or acquired using a variety of modalities. For example, during operation, the target registration module 102 registers target and reference objects by transforming at least one of computerized axial tomography imaging data, fiber optic thermometry imaging data, infrared thermography imaging data, magnetic resonance imaging data, magnetic resonance spectroscopy data, microwave thermography imaging data, microwave dielectric spectroscopy data, positron emission tomography imaging data, ultrasound reflectometry, spectroscopic imaging, visual imaging, infrared imaging, or single photon emission computed tomography imaging data into a reference coordinate frame (e.g., a coordinate frame associated with the patient, a coordinate frame associated with the energy delivery apparatus, etc.). In an embodiment, the target registration module 102 registers treatment targets and treatment focal regions using one or more transformations. Non-limiting examples of transformation includes affine transformations, fast three dimensional image transformations, geometric transformations, interpolating transformations, linear transformations, projective transformations, similarity transformations, or spline transformations. In an embodiment, the target registration module 102 detects and tracks anatomical targets and synchronizes treatment delivery to the one or more treatment focal regions 104 with a motion of the anatomical targets. For example, in an embodiment, the target registration module 102 includes a sensor component 110 that detects and tracks a relative spacing between an anatomical target and one or more treatment focal regions 104. In an embodiment, the target registration module 102 includes a sensor component 110 that determines a location, position, orientation, or the like of at least one anatomical target by monitoring a metabolic process. In an embodiment, the target registration module 102 includes a sensor component 110 having a plurality of sensors 112 that actively detect, track, or monitor one or more anatomical targets, biological structures, artificial surface markings, tattoos, nanoparticle fiducial markers, or the like. For example, in an embodiment, the target registration module 102 actively monitors (e.g., detects, tracks, etc.) an anatomical target located using at least one of computerized axial tomography, fiber optic thermometry, infrared thermography, magnetic resonance imaging, magnetic resonance spectroscopy, microwave thermography, microwave dielectric spectroscopy, positron emission tomography, ultrasound reflectometry, spectroscopic imaging, visual imaging, infrared imaging, or single photon emission computed tomography.

In an embodiment, the target registration module 102 includes a sensor component 110 that detects a location of a peripheral vascular bed, a biological structure, etc. using one or more imaging modalities. Non-limiting examples of imaging modalities include computerized axial tomography, fiber optic thermometry, infrared thermography, magnetic resonance imaging, magnetic resonance spectroscopy, microwave thermography, microwave dielectric spectroscopy, positron emission tomography, ultrasound reflectometry, spectroscopic imaging, visual imaging, infrared imaging, or single photon emission computed tomography. In an embodiment, the target registration module 102 includes a sensor component 110 that detects and tracks a location of a peripheral vascular bed relative to the movement of a transcutaneous energy delivery apparatus 202 using one or more imaging modalities. In an embodiment, the target registration module 102 registers the one or more treatment focal regions 104 with the at least one anatomical target based on a detected location of a peripheral vascular bed.

In an embodiment, the system 100 is configured to detect and track anatomical targets (e.g., via a plurality of sensors 112, etc.) and to generate at least one of a visual, an audio, a haptic, or a tactile representation indicative of a target registration status. For example, in an embodiment, the system 100 includes a target registration module 102 that generates, via one or more of a visual, audio, haptic, or a tactile representation, an indication that the one or more treatment focal regions 104 are registered with one or more treatment target and in position to deliver treatment. In an embodiment, the system 100 delivers a pro-apoptotic stimulus 103 to at least one treatment focal region 104 when registration indicates that the treatment focal region 104 coincides with a treatment target, and ceases delivery of the pro-apoptotic stimulus 103 when registration indicates that the treatment focal region 104 coincides with a non-treatment target.

In an embodiment, the target registration module 102 detects and tracks subsurface anatomical targets using subsurface thermography. For example, in an embodiment, the target registration module 102 includes one or more computing devices 114 that compare a detected thermograph associated with one or more subsurface anatomical targets to reference thermograph information (e.g., thermographic data, thermographic images, etc.) and generates treatment registration information based on the comparison. In an embodiment, the treatment registration information includes one or more of a location coordinate (e.g., a treatment location coordinate, etc.), a focal region dimension, a focal region depth, or a focal region beam axis direction. In an embodiment, the treatment registration information includes anatomical target identification information, anatomical target location information, anatomical target shape information, anatomical target dimension information, anatomical target distribution information, or point cloud information.

In an embodiment, the target registration module 102 includes at least one sensor component 110 having a thermal imaging system that acquires a thermograph of an anatomical target at one or more fields of view. For example, in an embodiment, the target registration module 102 includes one or more of an infrared imaging system, a thermography apparatus (e.g., a thermographic camera, an infrared thermographic camera, etc.) that measure temperatures within a biological subject at one or more fields of view.

In an embodiment, the target registration module 102 includes a computing device 114 configured to process sensor component 110 measurand information and to cause the storing of the measurand information in a data storage medium. In an embodiment, the target registration module 102 includes one computing device 114 operably coupled to one or more sensor components 110 such that the computing device 114 determines a sampling regimen based on measurand information communicated by the one or more sensor components 110. In an embodiment, the target registration module 102 includes one or more computing devices 114 that are operable to determine a spatial frequency spectrum of at least a first subset of pixels of the thermograph.

In an embodiment, the target registration module 102 includes one or more computing devices 114 that generate treatment registration information based on a comparison of the spatial frequency spectrum of the first subset of pixels to reference thermograph information. In an embodiment, the target registration module 102 includes one or more computing devices 114 operable to compare a detected dielectric profile associated with one or more subsurface anatomical targets to reference dielectric information and to generate treatment registration information based on the comparison. For example, in an embodiment, the target registration module 102 identifies groups of pixels in a thermograph indicative of at least one anatomical target imaged in the thermograph, and generates treatment registration information representative of a parameter associated with a location and a dimension of the one or more treatment focal regions 104. In an embodiment, the target registration module 102 includes a computing device 114 and a sensor component 110 configured to detect a biological structure. In an embodiment, the computing device 114 actuates an alignment of the one or more treatment focal regions 104 with the at least one anatomical target based on a detected measurand associated with the biological structure.

In an embodiment, the target registration module 102 includes at least one sensor component 110 including one or more sensor 112 that actively detects, tracks, or monitors one or more anatomical targets, biological structures, artificial surface markings, tattoos, nanoparticle fiducial markers, or the like. For example, in an embodiment, the target registration module 102 includes at least one sensor component 110 that acquires at least one of an acoustic measurement, an electromagnetic energy measurement, a pulse oximetry measurement, a thermal energy measurement, or the like and actively detects, tracks, or monitors one or more anatomical targets, biological structures, artificial surface markings, tattoos, nanoparticle fiducial markers, based on the acquired acoustic measurement.

Non-limiting examples of sensors 112 include acoustic transducers, electrochemical transducers, optical transducers, piezoelectrical transducers, or thermal transducers. Further non-limiting examples of sensors 112 include electrochemical detectors, fluorescent detectors, light scattering detectors, mass spectroscopy detectors nuclear magnetic resonance detectors, near-infrared detectors, radiochemical detectors, refractive index detectors, ultra-violet detectors, thermal energy detectors, or the like. Further non-limiting examples of sensors 112 include biosensors, detectors, refractive index detectors, blood volume pulse sensors, conductance sensors, electrochemical sensors, fluorescence sensors, force sensors, heat sensors (e.g., thermistors, thermocouples, or the like), high resolution temperature sensors, differential calorimeter sensors, optical sensors, goniometry sensors, potentiometer sensors, resistance sensors, respiration sensors, sound sensors (e.g., ultrasound), Surface Plasmon Band Gap sensor (SPRBG), physiological sensors, surface plasmon sensors, or the like. Further non-limiting examples of sensors 112 include chemical transducers, ion sensitive field effect transistors (ISFETs), ISFET pH sensors, membrane-ISFET devices (MEMFET), microelectronic ion-sensitive devices, potentiometric ion sensors, quadruple-function ChemFET (chemical-sensitive field-effect transistor) integrated-circuit sensors, sensors with ion-sensitivity and selectivity to different ionic species, or the like. Further non-limiting examples of sensors 112 can be found in the following documents: U.S. Pat. Nos. 7,396,676 (issued Jul. 8, 2008) and 6,831,748 (issued Dec. 14, 2004); each of which is incorporated herein by reference.

In an embodiment, the target registration module 102 includes one or more acoustic transducers, electrochemical transducers, optical transducers, piezoelectrical transducers, or thermal transducers. In an embodiment, the target registration module 102 includes one or more thermal detectors, photovoltaic detectors, or photomultiplier detectors. In an embodiment, the target registration module 102 includes one or more charge-coupled devices, complementary metal-oxide-semiconductor devices, photodiode image sensor devices, whispering gallery mode (WGM) micro cavity devices, or scintillation detector devices. In an embodiment, the one or more sensors 112 include one or more ultrasonic transducers.

In an embodiment, the target registration module 102 includes circuitry having one or more components operably coupled (e.g., communicatively, electromagnetically, magnetically, ultrasonically, optically, inductively, electrically, capacitively coupled, or the like) to each other. In an embodiment, circuitry includes one or more remotely located components. In an embodiment, remotely located components are operably coupled via wireless communication. In an embodiment, remotely located components are operably coupled via one or more receivers 116, transmitters 118, transceivers 120, or the like.

In an embodiment, circuitry includes, among other things, one or more computing devices 114 such as a processor (e.g., a microprocessor) 122, a central processing unit (CPU) 124, a digital signal processor (DSP) 126, an application-specific integrated circuit (ASIC) 128, a field programmable gate array (FPGA) 130, or the like, or any combinations thereof, and can include discrete digital or analog circuit elements or electronics, or combinations thereof. In an embodiment, circuitry includes one or more ASICs 128 having a plurality of predefined logic components 132. In an embodiment, circuitry includes one or more FPGAs 130 having a plurality of programmable logic components.

In an embodiment, circuitry includes one or more memories 134 that, for example, store instructions or data. Non-limiting examples of examples of one or more memories 134 include volatile memory (e.g., Random Access Memory (RAM) 136, Dynamic Random Access Memory (DRAM), or the like), non-volatile memory (e.g., Read-Only Memory (ROM) 138, Electrically Erasable Programmable Read-Only Memory (EEPROM), Compact Disc Read-Only Memory (CD-ROM), or the like), persistent memory, or the like. Further non-limiting examples of one or more memories 134 include Erasable Programmable Read-Only Memory (EPROM), flash memory, or the like. The one or more memories 134 can be coupled to, for example, one or more computing devices 114 by one or more instruction, data, or power buses.

In an embodiment, circuitry includes one or more computer-readable media drives 142, interface sockets, Universal Serial Bus (USB) ports, memory card slots, or the like, and one or more input/output components 144 such as, for example, a graphical user interface, a display 146, a keyboard 148, a keypad, a trackball, a joystick, a touch-screen, a mouse, a switch, a dial, or the like, and any other peripheral device. In an embodiment, circuitry includes one or more user input/output components 144 that are operably coupled to at least one computing device 114 to control (electrical, electromechanical, software-implemented, firmware-implemented, or other control, or combinations thereof) at least one parameter associated with transcutaneously delivering pro-apoptotic energy to the at least one treatment target.

In an embodiment, the system 100 includes one or more modules optionally operable for communication with one or more input/output components 144 that are configured to relay user output and/or input. In an embodiment, a module includes one or more instances of electrical, electromechanical, software-implemented, firmware-implemented, or other control devices. Such devices include one or more instances of memory 134; computing devices 114; antennas; power or other supplies; logic modules or other signaling modules; gauges or other such active or passive detection components; piezoelectric transducers, shape memory elements, micro-electro-mechanical system (MEMS) elements, or other actuators.

In an embodiment, the computer-readable media drive 142 or memory slot are configured to accept signal-bearing media (e.g., computer-readable memory media, computer-readable recording media, or the like). In an embodiment, a program for causing the system 100 to execute any of the disclosed methods can be stored on, for example, computer-readable recording media (CRMM) 146, signal-bearing media, or the like. Non-limiting examples of signal-bearing media include a recordable type media such as a magnetic tape, floppy disk, a hard disk drive, a memory device 134, or the like, as well as transmission type media such as a digital and/or an analog communication medium (e.g., a fiber optic cable, a waveguide, a wired communications link, a wireless communication link (e.g., receiver 116, transmitter 118, transceiver 120, transmission logic, reception logic, etc.), etc.). Further non-limiting examples of signal-bearing media include DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In an embodiment, the target registration module 102 includes circuitry having one or more databases 148. In an embodiment, a database 148 includes treatment registration information indicative of an alignment of at least one treatment focal region with a first adipose depot target. In an embodiment, a database 148 includes treatment registration information includes an anatomical target identification, anatomical target location, an anatomical target shape, an anatomical target dimension, an anatomical target distribution, or a point cloud associated with an anatomical target. In an embodiment, a database 148 includes at least one of a location coordinate (e.g., a treatment location coordinate, etc.), a focal region dimension, a focal region depth, or a focal region beam axis direction.

In an embodiment, a database 148 includes one or more heuristically determined parameters associated with at least one in vivo or in vitro determined metric. In an embodiment, a database 148 includes stored reference data 149 such as reference anatomical structure image data, computerized axial tomography imaging data, fiber optic thermometry imaging data, infrared thermography imaging data, magnetic resonance imaging data, magnetic resonance spectroscopy data, microwave thermography imaging data, microwave dielectric spectroscopy data, positron emission tomography imaging data, single photon emission computed tomography imaging data, ultrasound reflectometry, spectroscopic imaging, visual imaging, infrared imaging, or the like. In an embodiment, a database 148 includes 2-, 3-, or 4-dimensional human body structure atlas information.

In an embodiment, the target registration module 102 includes circuitry configured to detect at least one of energy absorption, energy reflection, or energy transmission spectra associated with, for example, an anatomical target. For example, in an embodiment, the target registration module 102 includes at least one sensor component 110 including one or more sensor 112, operably coupled by one or more computing devices 114, that detect at least one of energy absorption, energy reflection, or energy transmission spectra. In an embodiment, the target registration module 102 includes a computing device 114 configured to process sensor measurand information and configured to cause the storing of the measurand information in a data storage medium. In an embodiment, the target registration module 102 includes one or more computing devices 114 configured to compare a real-time detected measurand associated with one or more subsurface anatomical targets to reference subsurface anatomical target information, and to generate treatment registration information based on the comparison.

In an embodiment, the target registration module 102 includes a sensor component 110 configured to real-time motion-track the at least one anatomical target and the one or more treatment focal regions 104. For example, in an embodiment, the target registration module 102 acquires measurand data, via an array of sensors 112, allowing the target registration module 102 to track and store the motion of treatment focal regions 104. In an embodiment, the target registration module 102 includes a sensor component 110 configured to motion-track the movement of the at least one anatomical target relative to the one or more treatment focal regions 104. In an embodiment, the target registration module 102 is configured to motion-track the movement of the at least one anatomical target, and real-time position the one or more treatment focal regions 104 onto the at least one anatomical target.

In an embodiment, the target registration module 102 includes a sensor component 110 configured to detect one or more surface markings 140 (e.g., body surface markings, tattoos, machine-readable symbols, human readable symbols, quick response codes, moles, hair follicles, etc.). In an embodiment, the target registration module 102 registers one or more treatment focal regions 104 with at least one anatomical target relative to detected surface markings. In an embodiment, the target registration module 102 is configured to register the one or more treatment focal regions 104 with one or more surface markings 140, artificial surface markings, tattoos, nanoparticle fiducial markers, or the like and to generate treatment registration information.

In an embodiment, the target registration module 102 is configured to generate a treatment protocol based on the treatment registration information. For example, in an embodiment, the target registration module 102 registers a plurality of treatment focal region 104 locations and generates at least a first-in-time treatment protocol and a second-in-time treatment protocol based on the treatment registration information.

In an embodiment, the target registration module 102 is configured to register at least one non-treatment target and to generate non-treatment registration information. In an embodiment, the target registration module 102 is configured to register one or more non-treatment targets and one or more adipose depot targets 106 and to generate treatment and non-treatment registration information. In an embodiment, the system 100 includes a first configuration and a second configuration. In an embodiment, the first configuration is operable to deliver a pro-apoptotic energy stimulus 103 to at least one treatment focal region 104 when registration indicates that the treatment focal region 104 coincides with a treatment target, and the second configuration is operable to terminate energy delivery, prevent energy delivery, deactivate energy delivery, cease energy delivery, enter a non-delivery mode, etc., when registration indicates that the treatment focal region 104 coincides with a non-treatment target.

In an embodiment, the system 100 includes an apoptosis inducement module 150 configured to deliver a pro-apoptotic energy stimulus 103 to a plurality of treatment focal regions 104. For example, in an embodiment, the apoptosis inducement module 150 includes one or more energy emitters 152 that transcutaneously deliver a multi-focal pro-apoptotic energy stimulus 103 at a dose sufficient to induce programmed cell death, without substantially inducing necrosis, of adipocytes within one or more treatment focal regions 104.

In an embodiment, the apoptosis inducement module 150 determines a treatment protocol of pro-apoptotic energy and transcutaneously delivers pro-apoptotic energy to at least one treatment target according to the treatment protocol. For example, in an embodiment, the apoptosis inducement module 150 includes at least one computing device 114 configured to alter a duty cycle associated with a transcutaneous delivery of the pro-apoptotic energy based on a comparison of the detected measurand to a user-specific treatment protocol. In an embodiment, the apoptosis inducement module 150 includes at least one computing device 114 configured to alter a duty cycle associated with a transcutaneous delivery of pro-apoptotic energy based on a time variable behavior of a relative movement between the plurality of treatment focal regions 104 and the treatment target.

In an embodiment, the apoptosis inducement module 150 includes at least one computing device 114 configured to alter a duty cycle associated with a transcutaneous delivery of the pro-apoptotic energy based on a comparison of the detected measurand to a user-specific treatment protocol. In an embodiment, the apoptosis inducement module 150 includes at least one computing device 114 configured to alter a duty cycle associated with a transcutaneous delivery of the pro-apoptotic energy based on a time variable behavior of a relative movement between the plurality of treatment focal regions 104 and the treatment target. In an embodiment, the apoptosis inducement module 150 includes at least one computing device 114 configured to alter a duty cycle associated with a transcutaneous delivery of the pro-apoptotic energy based on a comparison of the detected measurand to a user-specific treatment protocol. In an embodiment, the apoptosis inducement module 150 includes at least one computing device 114 configured to alter a duty cycle associated with a transcutaneous delivery of the pro-apoptotic energy based on a time variable behavior of a relative movement between the plurality of treatment focal regions 104 and the treatment target.

In an embodiment, the apoptosis inducement module 150 includes one or more energy emitters 152 configured to transcutaneously deliver at least one of a pro-apoptotic electromagnetic energy stimulus, a pro-apoptotic electrical energy stimulus, a pro-apoptotic acoustic energy stimulus (e.g., a pro-apoptotic ultrasonic energy stimulus, a pro-apoptotic subsonic energy stimulus, a pro-apoptotic focused ultrasonic energy stimulus, etc.), or a pro-apoptotic thermal energy stimulus. In an embodiment, the apoptosis inducement module 150 includes one or more energy emitters 152 that concurrently or sequentially deliver one or more of a pro-apoptotic electromagnetic energy stimulus, a pro-apoptotic electrical energy stimulus, a pro-apoptotic acoustic energy stimulus, or a pro-apoptotic thermal energy stimulus.

Non-limiting examples of energy emitters 152 include electromagnetic energy emitters, acoustic energy emitters (e.g., sonic energy emitters, ultrasonic energy emitters, subsonic energy emitters, thermal energy emitters, or electrical energy emitters. Further non-limiting examples of energy emitters 152 include optical energy emitters and ultrasound energy emitters. Further non-limiting examples of energy emitters 152 include, electric circuits, electrical conductors, electrodes (e.g., nano- and micro-electrodes, patterned-electrodes, electrode arrays (e.g., multi-electrode arrays, micro-fabricated multi-electrode arrays, patterned-electrode arrays, or the like), electrocautery electrodes, or the like), cavity resonators, conducting traces, ceramic patterned electrodes, electro-mechanical components, lasers, quantum dots, laser diodes, light-emitting diodes (e.g., organic light-emitting diodes, polymer light-emitting diodes, polymer phosphorescent light-emitting diodes, microcavity light-emitting diodes, high-efficiency UV light-emitting diodes, or the like), arc flashlamps, incandescent emitters, transducers, heat sources, continuous wave bulbs, ultrasound emitting elements, ultrasonic transducers, thermal energy emitting elements, or the like. In an embodiment, the one or more energy emitters 152 include at least one two-photon excitation component. In an embodiment, the one or more energy emitters 152 include at least one of an exciplex laser, a diode-pumped solid state laser, or a semiconductor laser.

In an embodiment, the apoptosis inducement module 150 includes one or more energy emitters 152 configured to transcutaneously deliver one or more of a focused ultrasound energy stimulus or a focused electromagnetic energy stimulus. In an embodiment, the apoptosis inducement module 150 includes one or more energy emitters 152 configured to transcutaneously deliver a focused electromagnetic energy stimulus. In an embodiment, the apoptosis inducement module 150 includes one or more energy emitters 152 configured to transcutaneously deliver a focused microwave stimulus. In an embodiment, the apoptosis inducement module 150 includes at least one radiofrequency phased array configured to transcutaneously deliver a focused microwave stimulus at a dose sufficient to induce programmed cell death of adipocytes within the one or more treatment focal regions 104, without substantially inducing programmed cell death of non-adipocytes. In an embodiment, the apoptosis inducement module 150 includes one or more transducers configured to transcutaneously deliver a focused ultrasound energy stimulus at a dose sufficient to induce programmed cell death of adipocytes within the one or more treatment focal regions 104, without substantially inducing programmed cell death of non-adipocytes.

Further non-limiting examples of energy emitters 152 include radiation emitters, ion emitters, photon emitters, electron emitters, gamma emitters, or the like. In an embodiment, the one or more energy emitters 152 include one or more incandescent emitters, transducers, heat sources, or continuous wave bulbs. In an embodiment, the one or more energy emitters 152 include one or more laser, light-emitting diodes, laser diodes, fiber lasers, lasers, or ultra-fast lasers, quantum dots, organic light-emitting diodes, microcavity light-emitting diodes, or polymer light-emitting diodes. Further non-limiting examples of energy emitters 152 include electromagnetic energy emitters. In an embodiment, the apoptosis inducement module 150 includes one or more transducers.

In an embodiment, the system 100 includes one or more computing devices 114 that automatically control one or more of frequency, duration, pulse rate, duty cycle, or the like associated with a pro-apoptotic energy stimulus 103 generated by the one or more energy emitters 152 based on a sensed parameter. In an embodiment the system 100 includes one or more computing devices 114 that automatically control one or more of frequency, duration, pulse rate, duty cycle, or the like associated with the acoustic energy generated by the one or more energy emitters 152 based on a sensed parameter associated with a region within the biological subject.

In an embodiment, the apoptosis inducement module 150 includes one or more energy emitters 152 configured to transcutaneously deliver the pro-apoptotic energy stimulus 103 at a dose sufficient (e.g., a frequency sufficient, power sufficient, duration sufficient, intensity sufficient, duty cycle sufficient, at a pulse rate sufficient, etc.) to induce PCD of adipose tissue within the one or more treatment focal regions 104, without substantially inducing programmed cell death of non-adipose tissue. PCD (e.g., apoptosis, etc.) can be induced using a variety of methodologies and technologies including, for example, using acoustic energy, electricity, electromagnetic energy, thermal energy, pulsed electric fields, pulsed ultrasound, focused ultrasound, low intensity ultrasound, ultraviolet radiation, or the like. Localized heating therapy caused by the delivery of energy, for example via one or more energy emitters 152, can likewise induce PCD or necrosis of cells or tissue depending upon the temperature, exposure time, etc., experienced by the cells or tissue.

For example, in an embodiment, when actuated, the apoptosis inducement module 150 causes one or more energy emitters 152 that transcutaneously deliver a pro-apoptotic energy stimulus 103 at a dose sufficient to elevate a temperature of at least a portion of adipocytes within the one or more treatment focal regions 104. In an embodiment, localized heating therapy between 40° C. and 60° C. can result in disordered cellular metabolism and membrane function and in many instances, cell death (e.g., PCD). In general, at temperatures below 60° C., localized heating is more likely to induce PCD in cells, without substantially inducing necrosis. At temperatures greater than about 60° C., the likelihood of inducing coagulation necrosis of cells and tissue increases. Relatively small increases in temperature (e.g., a 3° C. increase) above the normal functioning temperature of a cell can cause apoptotic cell death. For example, temperatures ranging from 40° C. to 47° C. can induce cell death in a reproducible time and temperature dependent manner in cells normally functioning at 37° C.

Non-limiting examples of methodologies and technologies for inducing PCD can be found the following documents: Abdollahi et al., Apoptosis signals in Lymphoblasts Induced by Focused Ultrasound, FASEB Journal Express Article doi:10.1096/fj.04-1601fje (Published online Jul. 1, 2004); Ashush et al., Apoptosis Induction of Human Myeloid Leukemic Cells by Ultrasound Exposure, Cancer Res. 60: 1014-1020 (2000); Beebe et al., Nanosecond, High-intensity Pulsed Electric Fields Induce Apoptosis in Human Cells, The FASEB Journal express article 10.1096/.02-0859fje (Published online Jun. 17, 2003); Caricchio et al., Ultraviolet B Radiation-Induced Cell Death: Critical Role of Ultraviolet Dose in Inflammation and Lupus Autoantigen Redistribution, J. Immunol., 171: 5778-5786 (2003); Fabo et al., Ultraviolet B but not Ultraviolet A Radiation Initiates Melanoma, Cancer Res. 64 (18): 6372-376 (2004) ; Fent et al., Low lintensity Ultrasound-induced Apoptosis in Human Gastric Carcinoma Cells, World J Gastroenterol, 14(31):4873-879 (2008); Hall et al., Nanosecond Pulsed Electric Fields Induce Apoptosis in p53-Wildtype and p53-Null HCT116 Colon Carcinoma Cells, Apoptosis, 12(9):1721-31 (2007); and Rediske et al., Pulsed Ultrasound Enhances the Killing of Escherichia coli Biofilms by Aminoglycoside Antibiotics In vivo, Antimicrob. Agents Chemother., 44 (3): 771-72 (2000); each of which is incorporated herein by reference.

In an embodiment, apoptosis inducement module 150 concurrently or sequentially delivers one or more of a pulsed stimulus, a spatially patterned stimulus, a temporally patterned stimulus, or the like at a dose sufficient to induce programmed cell death, without substantially inducing necrosis, of adipocytes within the one or more treatment focal regions 104. In an embodiment, the apoptosis inducement module 150 includes one or more energy emitters 152 configured to transcutaneously deliver the pro-apoptotic energy stimulus 103 at a dose sufficient to induce programmed cell death of adipocytes within the one or more treatment focal regions, without substantially treatment focal regions 104 inducing programmed cell death of overlying tissue. For example, in an embodiment, the apoptosis inducement module 150 concurrently or sequentially delivers one or more of a pulsed stimulus, a spatially patterned stimulus, a temporally patterned stimulus, or the like at a dose sufficient to elevate a temperature of at least a portion of adipocytes within the one or more treatment focal regions 104, without substantially elevating a temperature of overlying tissue. In an embodiment, the apoptosis inducement module 150 includes one or more memories 134 having pro-apoptotic stimulus dose information stored thereon. In an embodiment, the apoptosis inducement module 150 includes at least one computing device 114 configured to alter a duty cycle associated with a transcutaneous delivery of the pro-apoptotic energy based on a comparison of pro-apoptotic stimulus dose information to a user-specific treatment protocol.

In an embodiment, the apoptosis inducement module 150 includes one or more energy emitters 152 that transcutaneously deliver a pro-apoptotic energy stimulus 103 at a dose sufficient to elevate a temperature of at least a portion of adipocytes within the one or more treatment focal regions 104 from about 3° C. to about 22° C., without substantially elevating a temperature of overlying tissue. In an embodiment, the apoptosis inducement module 150 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 3° C. to about 10° C., without substantially elevating a temperature of overlying tissue. In an embodiment, the apoptosis inducement module 150 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 3° C. to about 4° C., without substantially elevating a temperature of overlying tissue.

In an embodiment, the apoptosis inducement module 150 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 37° C. to less than about 60° C., without substantially elevating a temperature of overlying tissue. In an embodiment, the apoptosis inducement module 150 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 37° C. to less than about 47° C., without substantially elevating a temperature of overlying tissue. In an embodiment, the apoptosis inducement module 150 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 37° C. to less than about 45° C., without substantially elevating a temperature of overlying tissue. In an embodiment, the apoptosis inducement module 150 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 37° C. to less than about 42° C., without substantially elevating a temperature of overlying tissue. In an embodiment, the apoptosis inducement module 150 includes one or more energy emitters 152 configured to transcutaneously deliver a pro-apoptotic energy stimulus 103 at a dose sufficient to elevate a temperature of at least a portion of adipocytes within the one or more treatment focal regions 104 from greater than about 41° C. to less than about 63° C., without substantially elevating a temperature of overlying tissue.

In an embodiment, the apoptosis inducement module 150 includes one or more energy emitters 152 configured to transcutaneously deliver the pro-apoptotic energy stimulus 103 at a dose sufficient to induce programmed cell death of adipocytes within the one or more treatment focal regions 104, without substantially inducing programmed cell death of tissue proximate the one or more treatment focal regions 104. For example, in an embodiment, the apoptosis inducement module 150 includes one or more electromagnetic energy emitters, acoustic energy emitters, thermal energy emitters, or electrical energy emitter that are activated to transcutaneously deliver the pro-apoptotic energy stimulus 103 at a dose sufficient to induce programmed cell death of adipose tissue within the one or more treatment focal regions 104, without substantially inducing programmed cell death of non-adipose tissue.

In an embodiment, the apoptosis inducement module 150 is configured to deliver a pro-apoptotic energy stimulus 103 to at least one treatment focal region 104 according to a treatment cycle based on an induced apoptosis to necrosis comparison. For example, in an embodiment, the apoptosis inducement module 150 includes a plurality of energy emitters 152, that when activated, deliver a pro-apoptotic energy stimulus 103 to the at least one treatment focal region 104 according to a treatment cycle based on an estimated apoptosis:necrosis inducement ratio. In an embodiment, the apoptosis inducement module 150 determines the estimated apoptosis:necrosis inducement ratio based on a real-time measurand.

Apoptosis includes a sequence of morphological changes including blebbing, loss of cell membrane asymmetry and attachment, cell shrinkage including shrinkage of mitochondria and other organelles, nuclear fragmentation, chromatin condensation and DNA fragmentation. Apoptosis generates apoptotic bodies (e.g., membrane-bound cellular fragments containing cytoplasm and nuclear debris, etc.) that are taken up by neighboring phagocytic cells by the process of phagocytosis. The process of apoptosis includes a cascade of events mediated by caspases, a family of cysteine proteases. In an embodiment, apoptosis is artificially induced by exposing cells or tissue to pro-apoptotic biological, chemical, or energy stimuli. Necrosis encompasses a premature death of cells or tissue and can be triggered by infections, cancer, infarction, inflammation, toxins, or trauma. Necrosis is characterized by swelling of cellular organelles (e.g., mitochondria, endoplasmic reticulum, etc.) and the cytoplasm, followed by collapse of the plasma membrane and cellular lysis. Necrotic cells do not usually send out the same chemical signals to the immune system as do cells undergoing apoptosis and as such may not be cleared as readily from the system by phagocytosis. In general, defective or ineffective clearance of dying cells, whether apoptotic or necrotic, can contribute to persistence of inflammation, excessive tissue injury, and human pathologies, including systemic lupus erythematosus, cystic fibrosis, and chronic obstructive pulmonary disease. Engulfment of apoptotic cells is regulated by a highly redundant system of receptors and bridging molecules on the apoptotic cells and on the phagocytes.

The body normally loses more than a billion cells per day through the process of apoptosis. Apoptotic cells can be engulfed by “professional” phagocytes, non-limiting examples of which include neutrophils, monocytes, macrophages, dendritic cells, and mast cells. Further non-limiting examples of “professional” phagocytes include sinusoidal cells, osteoclasts, histiocytes, Kupffer cells, microglial cells, or Langerhans cells. Apoptotic cells can also be engulfed by “non-professional” neighboring cells, non-limiting examples of which include epithelial cells, endothelial cells, and fibroblasts. At the early stages of apoptosis, the dying cells release one or more signals that attract motile phagocytes to the proximity of the dying cells. These attractants can include triphosphate nucleotides, lysophosphatidylcholine, and chemokines. During the process of apoptosis, the cells redistribute phosphatidylserine, a phospholipid component of the cell membrane, from the cytoplasmic surface of the cell membrane to the extracellular surface of the cell membrane. Once in proximity to the dying cells the phagocytes interact with additional signals on the surface of the dying cells including, for example, phosphatidylserine, which aides in activating signaling pathways necessary for the process of phagocytosis.

Phagocytosis of the dying, apoptotic cell prevents the release of potentially toxic or immunogenic intracellular contents from the cell into the local environment. This is in contrast to necrotic cell death, where the unregulated release of material from dead cells, most notably intracellular antigens and nucleic acids, can cause strong inflammatory responses. In addition, phagocytes engaged in clearing apoptotic cells produce anti-inflammatory mediators that further suppress inflammation and facilitate the “immunologically silent” clearance of apoptotic cells. However, if apoptotic cells are not promptly cleared, the membrane integrity is lost over time and the cells can progress to secondary necrosis. See, e.g., Elliott & Ravichandron, J. Cell. Biol. 189:1059-1070 (2010); which is incorporated herein by reference.

In an embodiment, an induced apoptosis to necrosis comparison (e.g., a ratio of apoptotic cells to necrotic cells, an apoptosis:necrosis inducement ratio, a fraction of apoptotic cells, a fraction of necrotic cells, etc.) following treatment of one or more adipose depot with pro-apoptotic energy is determined by monitoring at least one measurand of apoptosis relative to at least one measurand of necrosis. In an embodiment, apoptosis is distinguished from necrosis based on morphological measurands, biochemical measurands, or measurands related to the interaction of the dying cells with phagocytes.

In an embodiment, apoptotic cells are differentiated from necrotic cells based on relative changes in cell morphology. In an embodiment, the apoptosis inducement module 150 determines the estimated apoptosis:necrosis inducement ratio based on relative changes in cell morphology. For example, apoptotic cells are morphologically smaller and denser than normal healthy cells, while necrotic cells tend to swell relative to normal healthy cells and then undergo cell lysis. Organelles in apoptotic cells (e.g., mitochondria, etc.) also appear to measurably shrink while those of necrotic cells appear to swell. In addition, apoptotic cells undergo chromatin condensation and formation of apoptotic bodies. In an embodiment, during operation, the apoptosis inducement module 150 generates an apoptosis:necrosis inducement ratio by monitoring relative changes in cell morphology.

In an embodiment, the apoptosis inducement module 150 includes at least one sensor component 110 that real-time images using time-lapse microscopy such as, for example, differential interference contrast (DIC), alone or in combination with epifluorescence optics, to monitor morphological changes associated with apoptosis versus necrosis. In an embodiment, these methods are used to follow specific morphological changes typical of apoptosis versus necrosis such as duration and onset of rounding up of cells, formation of apoptotic bodies, and chromatin condensation and to follow the timing and kinetics of these morphological changes. Krysko et al., Methods in Enzymology, 442:307-341 (2008); which is incorporated herein by reference.

In an embodiment, diffusion-weighted magnetic resonance (DWI) is used to assess morphological changes in apoptotic and/or necrotic cells. The technique of DWI takes advantage of differences between extra-, intra-, and transcellular diffusion of water molecules in a region of interest, e.g., at the site of energy treated adipose tissue. Because the majority of DWI signal relates to the extracellular space and tissue perfusion, any expansion or contraction of the extracellular environment due, for example, to cellular shrinking or swelling associated with apoptosis or necrosis will cause a loss of signal. As such DWI can be used to distinguish dying tissue from viable tissue. See, e.g., Blankenberg et al., Q. J. Nucl. Med. 47:337-348 (2003); which is incorporated herein by reference.

In an embodiment, water suppressed lipid proton spectroscopy, a magnetic resonance imaging technique, is used to detect cells undergoing apoptosis. Cells undergoing apoptosis have an associated increase in cytoplasmic neutral mobile lipid droplets composed of polyunsaturated fatty acids, cholesterol esters, and triglycerides. The resonance signal from neutral mobile lipids can be observed with standard water suppressed proton magnetic resonance spectroscopy. See, e.g., Blankenberg et al., Q. J. Nucl. Med. 47:337-348 (2003); which is incorporated herein by reference.

In an embodiment, the apoptosis inducement module 150 differentiates apoptotic cells from necrotic cells based on relative changes in cellular biochemistry, including for example, changes in the expression of biomolecules released from or on the surface of apoptotic cells versus necrotic cells. In an embodiment, the apoptosis inducement module 150 determines the estimated apoptosis:necrosis inducement ratio based on a measurand indicative of one or more markers of apoptosis. Non-limiting examples of markers of apoptosis include annexin V, apoptosis inducing factor, apolipoproteins C-1, Bax, truncated proapoptotic Bid (tBid), cytochrome c, Bcl-2, BM-1/JIMRO, BV2, caspase-1, caspase-3, CD95, cleaved cytokeratin-18, clusterin, histone, NAPO (negative in apoptosis), M30, OX-42 IR, p41, p53, plasminogen activator inhibitor 2, poly ADP ribose polymerase (PARP), 120 kDa breakdown product of spectrin, survivin, tissue polypeptide antigen, tissue transglutaminase and ubiquitin.

In an embodiment, the system 100 determines the estimated apoptosis:necrosis inducement ratio based on a measurand indicative of the binding of annexin V to apoptotic cells. Annexin V is an endogenous mammalian protein with nanomolar affinity for phosphatidylserine. During the early stages of apoptosis, phosphatidylserine migrates from the inner leaflet of the cell membrane to the outer leaflet of the cell membrane. In an embodiment, an increased concentration of phosphatidylserine on the surface of apoptotic cells is measured using annexin V. The number of annexin V binding sites per cell increases approximately 100 to 1000 fold during the apoptotic process and as such can be used to measure early to intermediate phases of apoptosis (e.g., before extensive DNA fragmentation, etc.). Another example of a marker of apoptosis that binds to phosphatidylserine includes but is not limited to the C2 domain of synaptotagmin I. See, e.g., Guo et al., J. Exp. Clin. Canc. Res., 38:136, 2009 and Blankenberg et al., Q. J. Nucl. Med., 47:337-348, 2003, which are incorporated herein by reference. In an embodiment, apoptosis is assessed by measuring the activation, activity, etc. of one or more caspases. Caspases are aspartate-specific cysteine proteases activated as part of the apoptotic process. Caspases 2, 3, and 6-10 are specifically involved in the apoptotic process although caspases 1, 4, 5, and 11-14 may also play a role. In an embodiment, the system 100 images apoptotic cells in vivo by using tracers which act as inhibitors of one or more caspases. For example, in an embodiment, the apoptosis inducement module 150 uses measurand information associated with benzyloxycarbonyl-Val-Ala-DL-Asp(O-methyl)-fluoromethyl ketone, a pan caspase inhibitor, is labeled with ¹³¹I to identify apoptosis in vivo. See, e.g., Blankenberg et al., Q. J. Nucl. Med. 47:337-348 (2003); which is incorporated herein by reference.

Electromagnetic energy in the near-infrared (NIR) region (e.g., ranging from about 700 nm to about 1000 nm) can significantly traverse through tissue. In an embodiment, the apoptosis inducement module 150 includes one or more near-infrared electromagnetic energy sensors that employ near-infrared imaging techniques and methodologies (e.g., near-infrared fluorescence (NIRF), etc.) to detect and visualize, for example, fluorescent probes in vivo. In an embodiment, the amount of necrotic tissue versus apoptotic tissue following exposure to pro-apoptotic energy stimuli is assessed by monitoring fluorescent probes associated with cleavage of the peptide by endogenous caspases released in response to apoptosis. In an embodiment, a pan caspase inhibitor is fluorescently labeled with a near-infrared dye such as, for example, DyLight® 690 or DyLight®747 or with carboxyfluorescein or sulforhodamine B (See, e.g., Griffin et al., Technol. Canc. Res. Treatment, 6:651-654 (2007); which is incorporated herein by reference). In an embodiment, reversible or irreversible inhibitors of caspase activation are generated by coupling caspase-specific peptides to certain aldehyde, nitrile, or ketone compounds. Non-limiting examples of caspase inhibitors that fall into this category include Z-DEVD-FMK, Z-IETD-FMK, Z-LEHD-FMK, Z-VAD-FMK, Z-YVAD-FMK, Z-LEED-FMK, Z-WEHD-FMK, NP-DEVE-AOMK, NP-LETD-AOMK, and NP-LEHD-AOMK, a number of which are available from commercial sources (from, e.g., R&D Systems, Minneapolis, MN; EMD4Biosciences, Gibbstown, N.J.). See, e.g., Berger et al., Cell Res. 16:961-963, 2006 which is incorporated herein by reference. Further non-limiting examples of caspase inhibitors include IDN-6556 ((3-{2-[(2-tert-butyl-phenylaminooxalyl)-amino]-propionylamino}-4-oxo-5-(2,3,5,6-tetrafluoro-phenoxy)-pentanoic acid)) and anilinoquinazolines. See, e.g., Scott, et al., JPET, 304:433-440 (2003); which is incorporated herein by reference. Further non-limiting examples of caspase inhibitors are available from commercial sources such as, for example, Santa Cruz Biotechnology, Inc. (Santa Cruz, Calif.).

In an embodiment, apoptotic cells are imaged in vivo by using tracers which act as substrates of one or more caspases. For example, in an embodiment, a caspase cleavable peptide is labeled with a fluorescent probe that becomes active upon cleavage of the peptide by endogenous caspases released in response to apoptosis. See, e.g., Messerli et al., Neoplasia, 6:95-105 (2004); which is incorporated herein by reference. Non-limiting examples of peptides or peptide mimetics for use as caspase substrates include DEVD, I/LETD, LEHD, YEVD, WEHD, YVAD, DMQD, and VEID, analogues of which are available from commercial sources (from, e.g., Sigma-Aldrich, St. Louis, Mo.).

In an embodiment, the amount of necrotic tissue versus apoptotic tissue following exposure to pro-apoptotic energy stimuli is assessed by comparing image analysis generated using markers of necrosis with that generated using markers of apoptosis to generate an apoptosis:necrosis inducement ratio. In an embodiment, the system 100 determines an apoptosis:necrosis inducement ratio following exposure to pro-apoptotic energy by comparing time-series data generated using markers of necrosis with that generated using markers of apoptosis. For example in an embodiment, one of the target registration module 102 or the apoptosis inducement module 150 determines an apoptosis:necrosis inducement ratio following exposure to pro-apoptotic energy by comparing before and after images generated using markers of necrosis with that generated using markers of apoptosis. Generally, necrotic cells have a characteristic loss of cell membrane integrity. Under these conditions, otherwise cell impermeant agents are able to enter necrotic cells. Similarly, components of the cell (e.g., cytoplasm, DNA, etc.), which are not normally excreted from the cell, can be found in the extracellular space.

In an embodiment, the system 100 monitors necrosis by using one or more agents that stain nuclei or other cell organelles of cells that have lost membrane integrity. For example, in an embodiment, 1,10-dioctadecyl-3,3,30,30-tetramethylindocarbocyanine perchlorate (DiI) is used to monitor necrotic cells in vivo. See, e.g., Cordeiro et al., Cell Death Disease, 1, e3 (2010). In an embodiment, necrosis is monitored using one or more cell impermeant DNA binders such as, for example, propidium iodide, ethidium bromide, Hoechst stains, or 7-amino-actinomycin D. In an embodiment, Hoechst-IR (combination of DNA binding Hoechst dye and IR-786) is used to image loss of cell membrane integrity in necrotic tissue using near-infrared optical fluorescence imaging. See, e.g., Dasari et al., Org. Letters, 12:3300-3303 (2010); which is incorporated herein by reference.

In an embodiment, necrosis is monitored by assessing formation of one or more metabolic substrates normally formed in intact cells, but leaking out of necrotic cells that have lost membrane integrity. For example, in an embodiment, one of the target registration module 102 or the apoptosis inducement module 150 images necrotic cells in vivo by monitoring the conversion of [1,4-¹³C₂]fumarate to [1,4-¹³C₂]malate using magnetic resonance spectroscopy. See, e.g., Gallagher, et al., Proc. Natl. Acad. Sci., U.S.A., 106:19801-19806 (2009); which is incorporated herein by reference. Similarly, in an embodiment, one of the target registration module 102 or the apoptosis inducement module 150 determines necrosis or loss of cellular membrane integrity by measuring lactate dehydrogenase activity by monitoring the conversion of [1-¹³C]pyruvate into [1-¹³C]lactate using magnetic resonance spectroscopy. See, e.g., Witney et al., Neoplasia, 11:574-582 (2009); which is incorporated herein by reference.

In an embodiment, necrosis is monitored by assessing release of high mobility group box 1 protein (HMGB-1). HMGB-1 is an architectural chromatin-binding factor that is released from necrotic cells but remains bound to DNA in apoptotic cells. In an embodiment, necrosis is monitored by assessing release of heat shock protein 72 (Hsp72). Cyclophilin A release can also be used as a marker of necrotic cell death. See, e.g., Krysko et al., Methods Enzymology 442:307-341 (2008); Williams & Ireland, J. Leukoc. Biol. 83:489-492 (2008); and Christofferson & Yuan, Cell Death Differentiation, 17:1942-1943 (2010); each of which is incorporated herein by reference.

In an embodiment, apoptosis or necrosis is measured using one or more dyes that are sensitive to mitochondrial membrane potential, such as for example tetramethylrhodamine methyl ester perchlorate or JC-1. In an embodiment, apoptosis or necrosis is measured using one or more dyes for measuring reactive oxygen species such as for example dihydrorhodamine 123. In both cell death pathways, the membrane potential of the mitochondria eventually drops and cells start to produce reactive oxygen specifies (ROS). A reduction in dye intensity indicates reduction in mitochondrial function and an indication that the cell is dead or dying.

In an embodiment, one of the target registration module 102 or the apoptosis inducement module 150 determines necrosis versus apoptosis by measuring release of cytokeratin 18. During apoptotic death, cytokeratin 18 released from cells is cleaved at Asp396 by a caspase. In comparison, during necrotic death, soluble cytokeratin 18 is released from the cell. Antibodies that distinguish between the caspase-cleaved form of cytokeratin 18 and the soluble form of cytokeratin 18 is used to determine a ratio that reflects the type of cell death. For example, induction of apoptosis will result in the release of caspase-cleaved cytokeratin 18 and thus a relatively high caspase-cleaved:soluble cytokeratin 18 ratio. In contrast, induction of necrosis will result almost exclusively in the release of soluble cytokeratin 18 and therefore a relatively low caspase-cleaved:soluble cytokeratin 18 ratio. See, e.g., Krysko et al., Methods Enzymology 442:307-341 (2008); which is incorporated herein by reference.

In an embodiment, the system 100 determines apoptosis or necrosis of adipose tissue by transcutaneously detecting one or more one or more markers of apoptosis or necrosis. In an embodiment, the ratio of annexin and propidium iodide staining is used to monitor apoptosis versus necrosis. Cells with a low level of staining with annexin or propidium iodide are considered normal cells. Cells stained primarily with annexin are considered in the early stages of apoptotis. Cells stained with both annexin and propidium iodide are entering the later stages of apoptosis. And cells stained with only propidium iodide are considered dead. The progressive changes in the distribution of staining with annexin or propidium iodide is used to indicate what proportion of the cell population is becoming apoptotic and dying. See, e.g., Lin et al., Int. J. Obesity, 28:1535-1540 (2003); which is incorporated herein by reference.

In an embodiment, the system 100 determines apoptosis or necrosis of adipose tissue treated with a pro-apoptotic energy stimulus 103, in vivo, using one or more imaging modalities in combination with one or more labeled agents that bind to or interact with one or more markers of apoptosis or necrosis. Non-limiting examples of labeled agents include an antibody, aptamer, substrate, inhibitor or other entity that binds to or interacts with one or more endogenous markers of apoptosis or necrosis. In some embodiments, the labeled agent is a non-specific cell impermeant agent capable of entering dead or dying cells in which membrane integrity has been disrupted. Non-limiting examples of labels for use in labeling agents capable of binding to or interacting with markers of apoptosis or necrosis include fluorescent labels, magnetic labels, microbubbles, etc. Non-limiting examples of imaging modalities for use in imaging the distribution of labeled agents in apoptotic or necrotic cells and tissue following administration to a subject include positron emission tomography (PET), single photon emission computed tomography (SPECT), fluorescence molecular tomography, ultrasound, magnetic resonance imaging, ultrasound reflectometry, spectroscopic imaging, visual imaging, infrared imaging, etc. In an embodiment, the system 100 includes a plurality of sensors 112 that monitor one or more measurands associated with a level of necrosis of the one or more adipose depot targets 106 caused by a delivery of the pro-apoptotic energy stimulus 103, and a computing device 114 operably coupled to the plurality of sensors and the apoptosis inducement module 150.

In an embodiment, the labeled agent capable of binding to or interacting with one or more markers of apoptosis or necrosis is labeled with one or more radiolabels. As an example, annexin V is labeled with ⁹⁹mTc by first modifying annexin V at lysine groups with a linker, e.g., succinimidyl (6-hydrazinopyridine-3-carboxylic acid), and then conjugating the modified annexin V to a ⁹⁹mTc derivative, e.g., ⁹⁹mTc-pertechnetate. Similarly, methods can be used to conjugate ⁹⁹mTc to other markers of apoptosis. The resulting radiolabeled annexin V is administered to a subject, and its binding to phosphotidylserine on the surface of apoptotic cells is assessed using, for example, a conventional gamma camera or SPECT. (Blankenberg, J. Nucl. Med. 49:81S-95S (2008); which is incorporated herein by reference).

In an embodiment, the labeled agent capable of binding to or interacting with one or more markers of apoptosis or necrosis is radiolabeled with iodine-123, iodine-125 or fluorine-18 and imaged using single photon emission computed tomography (SPECT) imaging or positron emission tomography (PET) to assess apoptosis. For example, annexin V is radiolabeled with ¹⁸F using N-succinimidyl-4-¹⁸F-fluorobenzoic acid. The radiolabeled annexin V is administered intravenously and its distribution in apoptotic tissue assessed using PET. (Yagle et al., J. Nucl. Med. 46:658-666 (2005); which is incorporated herein by reference). Further non-limiting examples of radioisotopes used in medical imaging include carbon-11, nitrogen-13, oxygen-15, iodine-125, iodine-131, strontium-89, and indium-111. Further non-limiting examples of radiolabeled agents capable of binding to or interacting with one or more markers of apoptosis or necrosis include 4-[¹⁸F]Fluorobenzoyl-annexin V, ^{99m}Tc-Labeled hydrazinonicotinamide-cysteine-annexin A5, ¹¹¹In-Diethylenetriaminepentaacetic acid-polyethylene glycol-annexin V, ¹²³I-Annexin V, ¹²⁴I-Annexin V, (5)-1-(4-2-[¹¹C]Methoxybenzyl)-5-(2-phenoxymethyl-pyrrolidine-1-sulfonyl)-1H-indole-2,3-dione (caspase inhibitor), and (S)-1-(4-(2-[¹⁸F]Fluoroethoxy)benzyl)-5-[1-(2-methoxymethyl-pyrrolidiny)sulfonyl]-1H-indole-2,3-dione (caspase 3 inhibitor), all of which are described in the Molecular Imaging and Contrast Agent Database (MICAD; http://micad.nih.gov; Bethesda (Md.): National Center for Biotechnology Information (US)).

In an embodiment, the labeled agent capable of binding to or interacting with one or more markers of apoptosis or necrosis can include one or more fluorescent dyes. For example, annexin V is labeled with Cy5.5 (from Amersham Biosciences—GE Healthcare, Piscataway N.J.), administered intravenously to a subject, and binding to apoptotic cells monitored using in vivo fluorescence molecular tomography. Non-limiting examples of fluorescent dyes for use in labeling agents capable of binding to or interacting with markers of apoptosis or necrosis include one or more fluorescent dyes commonly used for diagnostic fluorescence imaging including fluorescein (FITC), indocyanine green (ICG) and rhodamine B.

Non-limiting examples of other fluorescent dyes for use in fluorescence imaging include cyanine dyes (e.g., Cy5, Cy5.5, or Cy7 (Amersham Biosciences, Piscataway, N.J., USA)) and Alexa Fluor dyes (e.g., Alexa Fluor 633, Alexa Fluor 635,

Alexa Fluor 647, Alexa Fluor 660, Alexa Fluor 680, Alexa Fluor 700, or Alexa Fluor 750 (Molecular Probes-Invitrogen, Carlsbad, Calif., USA)). See, e.g., U.S. Pat. App. No. 2005/0171434, incorporated herein by reference. Additional fluorophores include IRDye800, IRDye700, and IRDye680 (LI-COR, Lincoln, Nebr., USA), NIR-1 and 105-OSu (Dojindo, Kumamoto, Japan), La Jolla Blue (Diatron, Miami, Fla., USA), FAR-Blue, FAR-Green One, and FAR-Green Two (Innosense, Giacosa, Italy), DY-731, DY-783 (from, e.g., Dyomic GmbH, Germany), ADS 790-NS and ADS 821-NS (American Dye Source, Montreal, Calif.), NIAD-4 (ICx Technologies, Arlington, Va.). Other fluorescing agents include BODIPY-FL, europium, green, yellow and red fluorescent proteins, and luciferase. Non-limiting examples of near-infrared quantum dots are used for deep tissue imaging include CdTeSe/CdS, InAs/InP/ZnSe, CdTe/CdSe, PbS, CdHgTe, CdTe/CdS, and CdTe/CdSe/ZnS (Aswathy et al., Anal. Bioanal. Chem. 397:1417-1435 (2010); which is incorporated herein by reference).

Further non-limiting examples of other agents useful for assessing apoptosis or necrosis using fluorescence imaging techniques can be found in the Molecular Imaging and Contrast Agent Database (MICAD). Bethesda (Md.): National Center for Biotechnology Information (US) and include annexin B12 Cys101, Cys260-N,N′-dimethyl-N-(iodoacetyl)-N′-(7-nitrobenz-2-oxa-1,3-diazol-4-yl)ethylenediamine, Cy5-Glu-Pro-Asp-acyloxymethyl ketone, IRDye 700DX-Labeled annexin V, Pyro-Gly-Asp-Glu-Val-Asp-Gly-Ser-Gly-Lys(BHQ3).

In an embodiment, labeled agents that bind to, or interact with, one or more markers of apoptosis or necrosis are conjugated to magnetic particles for use as targeted magnetic resonance contrast agents in magnetic resonance imaging of treated adipose tissue. Non-limiting examples of magnetic resonance contrast agents can include paramagnetic contrast agents based on chelates of gadolinium or super-paramagnetic contrast agents that use mono- or polycrystalline iron oxide. Non-limiting examples of protein-based platforms with gadolinium include albumin, poly-L-lysine, avidin, and direct monoclonal antibody conjugates. Polyamidoamine dendrimers and cross-linked liposomes can also be used as carriers of gadolinium as well as modified with target specific antibodies. Iron-oxide based contrast agents usually consist of a magnetic core embedded in a polymer coating such as for example dextran or other polysaccharide, polyethyleneglycol, See, e.g., Artemov J. Cell. Biochem. 90:518-524 (2003); which is incorporated herein by reference. In an embodiment, annexin V or other marker of apoptosis or necrosis is conjugated to liposomes coated with gadolinium deithylenetriamine penta-acetate (Gd-DTPA) and used for magnetic resonance imaging of apoptotic or necrotic cells. See, e.g., Korngold et al., Heart Fail. Rev., 13:163-173 (2008); which is incorporated herein by reference. In another example, the C2 domain of synaptotagmin I is conjugated to iron oxide particles and used for magnetic resonance imaging of apoptotic cells. See, e.g., Blankenberg et al., Q. J. NucL Med., 47:337-348 (2003); which is incorporated herein by reference.

In an embodiment, labeled agents for binding to or interacting with one or more markers of apoptosis or necrosis include microbubbles modified with one or more agents that bind to or interact with one or more markers of apoptosis or necrosis. In an embodiment, modified microbubbles are used as target contrast agents for ultrasound imaging. For example, phospholipid microbubbles filled with ocatfluoropropan and modified on the surface with avidin are conjugated with biotinylated annexin V to form microbubbles labeled with annexin V. In an embodiment, microbubbles are administered to the subject and their distribution in apoptotic tissue imaged using targeted ultrasound. Similar methods can be used to conjugate other markers of apoptosis to microbubbles. See, e.g., Min et al., J. Cardiovasc. Ultrasound, 18:91-97 (2010); which is incorporated herein by reference.

In an embodiment, the agent used for assessing apoptosis or necrosis includes a plurality of imaging markers. Non-limiting examples of imaging markers include annexin A5-quantum dot-DTPA-gadolinium and Annexin V-cross-linked iron oxide-Cy5.5 for use in both optical fluorescence imaging and magnetic resonance imaging.

In an embodiment, adipose tissue is imaged before or after treatment with one or more imaging modality. Non-limiting examples of imaging modalities include magnetic resonance imaging (MRI); computed tomography, whole body scan with low-radiation dual energy x-ray absorptiometry (DXA), positron emission tomography, ultrasound, and acoustic radiation force impulse (ARFI) imaging. For example, PET is used in combination with systemically administered [18F]-2-fluoro-2-deoxy-D-glucose to measure glucose uptake in adipose tissue. See, e.g., Lunati et al., Int J Obesity, 25:457-461, 2001; Shen et al., Obes. Res. 11:5-16, 2003; Fahey et al., Utrason. Imaging, 28:193-210, 2006; which are incorporated herein by reference.

On a normal day, the human body is able to clear 10 billion cells to accommodate new cells generated by mitosis. Apoptotic adipocytes are cleared by surrounding phagocytes or phagocytes that have been recruited to the site of apoptosis. Both adipocytes and preadipocytes are capable of undergoing apoptosis in response to stimuli. Preadipocytes may be more susceptible to apoptosis than fully differentiated adipocytes. See, e.g., Sorisky et al., Int. J. Obesity, 24, Supp14:S3-S7 (2000); which is incorporated herein by reference. It is anticipated that triglycerides associated with the apoptotic adipocytes will be taken up by neighboring phagocytes during the process of phagocytosis. Triglycerides that are otherwise not taken up by neighboring phagocytes may be released into the interstitial fluid compartments of the surrounding adipose tissue. There the triglycerides will either be incorporated into very low density lipoprotein particles (VLDL) or low density lipoprotein particles (LDL) or be hydrolyzed by lipoprotein lipase to free fatty acids and glycerol. VLDL and LDL are transported to the liver and the associated triglycerides are hydrolyzed to fatty acids and glycerol. The relatively insoluble fatty acids are carried in the blood by albumin and eventually transported to the liver or other tissues for use as building blocks or for energy expenditure.

In an embodiment, an induced apoptosis to necrosis comparison following treatment of one or more adipose depot with pro-apoptotic energy is determined by monitoring at least one measurand associated with necrosis. In an embodiment, the at least one measurand associated with necrosis includes at least one measurand indicative of an inflammatory state. Non-limiting examples of measurands indicative of an inflammatory state include temperature, edema, inflammatory markers, or inflammatory cells.

In an embodiment, an induced apoptosis to necrosis comparison following treatment of one or more adipose depot with pro-apoptotic energy is determined by monitoring localized changes in body temperature. Acute inflammation caused by injury to tissue is accompanied by an increase in blood flow to the site of injury. The increased blood flow to the site of injury causes a measureable increase in local body temperature as well as the redness associated with inflammation. The release of chemical mediators of inflammation can also contribute to the rise in temperature at the site of injury. Non-limiting examples of methods for monitoring changes in local body temperature include infrared imaging, as described, for example, in Jones IEEE Transactions on Medical Imaging 17:1019-1027 (1998), which is incorporated herein by reference.

In an embodiment, an induced apoptosis to necrosis comparison following treatment of one or more adipose depot with pro-apoptotic energy is determined by monitoring edema associated with inflammation. An increase in the permeability of blood vessels at the site of tissue injury results in leakage of plasma proteins and fluid (edema) into the interstitial space, leading to tissue swelling. Non-limiting examples of methods for monitoring edema include near-infrared fluorescence imaging, radioisotopic imaging, magnetic resonance imaging, x-ray computed tomography, positron emission tomography, or visible and near-infrared spectral imaging as described, for example, in Kenne and Lindbom, Thromb. Haemost. 105:783-789 (2011) and in Stamatas et al., J. Invest. Dermatol. 126:1753-1760 (2006) ; each of which is incorporated herein by reference.

In an embodiment, an induced apoptosis to necrosis comparison following treatment of one or more adipose depot with pro-apoptotic energy is determined by monitoring one or more of inflammatory markers in the tissue. Inflammatory markers are released and/or activated in response to an inflammatory stimulus, e.g., tissue injury and can include both mediators and inhibitors of inflammation. Non-limiting examples of inflammatory markers include interferons, interleukins, tumor necrosis factor (TNF), granulocyte colony stimulating factor (G-CSF), granulocyte-macrophage colony stimulating factor (GM-CSF), macrophage colony-stimulating factor (M-CSF), gelsolin, erythropoietin (EPO), thrombopoietin (TPO), chemotactic cytokines (chemokines) and chemokine like molecules. Other non-limiting examples of inflammatory markers include anaphylatoxin fragments C3a, C4a, and C5a from the complement pathway, leukotrienes, prostaglandins, growth factors, soluble receptors to tumor necrosis factor receptor (sTNFr), soluble interleukin receptors sIL-1r and sIL-2r, C-reactive protein, CD11b, histamine, serotonin, apolipoprotein A1, β2-microglobulin, bradykinin, D-dimer, endothelin-1, eotaxin, factor VII, fibrinogen, globins, insulin, leptin, lymphotactin, von Willebrand factor, thromboxane, platelet activating factor (PAF), immunoglobulins, endotoxins, or exotoxins.

In an embodiment, an induced apoptosis to necrosis comparison following treatment of one or more adipose depot with pro-apoptotic energy is determined by monitoring the accumulation of one or more types of inflammatory cells in the tissue. Inflammatory cells contribute to the inflammatory response by releasing pro-inflammatory and anti-inflammatory mediators and as scavengers tasked to remove potentially damaging elements released as a result of the inflammatory response. Non-limiting examples of inflammatory cells include neutrophils, eosinophils, basophils, lymphocytes, monocytes, mast cells, macrophages, or dendritic cells.

Non-limiting examples of methods for in vivo imaging of inflammatory markers and/or inflammatory cells include positron emission tomography (PET), single-photon emission computed tomography, optical imaging, magnetic resonance imaging, and/or ultrasound imaging using one or more targeted radiolabeled, fluorescent, magnetic, and/or microbubble probes. For example, the influx of neutrophils and/or eosinophils at a site of inflammation can be monitored by positron emission tomography in combination with one or more radiolabeled antibodies directed against one or more surface antigens, e.g., CD-15 or CD-66. Other non-limiting examples of methods for in vivo imaging of inflammatory markers and/or inflammatory cells, or the like may be found in, for example, Imam et al., Radiotracers for imaging of infection and inflammation—A Review, World J. Nucl. Med. 40-55 (2006), Pirko et al., FASEB 18:179-181 (2004), Gessner & Dayton, Mol. Imaging 9:117-127 (2010); each of which is incorporated herein by reference.

In an embodiment, the apoptosis inducement module 150 is configured to alter the treatment cycle associated with a delivery of the pro-apoptotic energy stimulus 103 to the at least one treatment focal region 104 based on the estimated apoptosis:necrosis inducement ratio. In an embodiment, the apoptosis inducement module 150 is configured to alter the treatment cycle associated with a delivery of the pro-apoptotic energy stimulus 103 to the at least one treatment focal region 104 based on a probability of inducing apoptosis of one or more depot targets within the at least one treatment focal region, a probability of inducing necrosis of one or more depot targets within the at least one treatment focal region, or a combination thereof In an embodiment, the apoptosis inducement module 150 determines the estimated apoptosis:necrosis inducement ratio based on previous-in-time treatment information.

In an embodiment, the system 100 estimates an apoptosis:necrosis inducement ratio based on the one or more measurands of necrosis, and alters the duty cycle associated with a delivery of the pro-apoptotic energy based on the estimated apoptosis:necrosis inducement ratio. For example, in an embodiment, the apoptosis inducement module 150 is configured to alter a duty cycle associated with a delivery of the pro-apoptotic energy stimulus 103 to the plurality of treatment focal regions 104 based on at least one measurand associated with the one or more adipose depot targets 106.

In an embodiment, the apoptosis inducement module 150 alters a duty cycle associated with the delivery of a pro-apoptotic energy stimulus 103 based on one or more comparisons between at least one measurand and a user-specific treatment protocol. For example, in an embodiment, the apoptosis inducement module 150 varies one or more parameters associated with the delivery of the pro-apoptotic energy stimulus 103 when the at least one measurand meets or exceeds a threshold level. In an embodiment, the apoptosis inducement module 150 varies one or more parameters associated with the delivery of the pro-apoptotic energy stimulus 103 when the at least one measurand satisfies a target criterion. For example, in an embodiment, the apoptosis inducement module 150 varies a duty cycle associated with the delivery of the pro-apoptotic energy stimulus 103 in response to an estimated apoptosis:necrosis inducement ratio of the one or more adipose depot targets 106.

In an embodiment, the system 100 includes a plurality of sensors 112 configured to acquire a temperature profile of the one or more adipose depot targets 106 at a plurality of time periods, and at one or more fields of view. For example, in an embodiment, the target registration module 102 on-limiting or more of an infrared imaging system, a thermography apparatus (e.g., a thermographic camera, an infrared thermographic camera, etc.) that measure temperatures within a biological subject at one or more fields of view. In an embodiment, the system 100 acquires a thermograph of an anatomical target, via a thermo-imaging device, at one or more fields of view. In an embodiment, a computing device 114 is operably coupled to a plurality of sensors 112 and the apoptosis inducement module 150, and is configured to alter the duty cycle associated with a delivery of the pro-apoptotic energy based on a comparison of a detected temperature profile to a target temperature profile.

In an embodiment, the system 100 includes a computing device 114 operably coupled to the target registration module 102 and the apoptosis inducement module 150, and configured to alter the duty cycle associated with a delivery of the pro-apoptotic energy based on at least one measurand indicative of a presence of necrosis or apoptosis.

In an embodiment, the target registration module 102 includes one or more sensors 112 configured to detect the one or more adipose depot targets 106 and to generate real-time detected adipose depot information. Non-limiting examples of adipose depot information includes at least one of a thermo profile, a dielectric profile, or an impedance profile. Further non-limiting examples of adipose depot information includes at least one of computerized axial tomography imaging data, fiber optic thermometry imaging data, infrared thermography imaging data, magnetic resonance imaging data, magnetic resonance spectroscopy data, microwave thermography imaging data, microwave dielectric spectroscopy data, positron emission tomography imaging data, ultrasound reflectometry, spectroscopic imaging, visual imaging, infrared imaging, or single photon emission computed tomography imaging data. Further non-limiting examples of adipose depot information include at least one of adipose depot location information, adipose depot composition information, adipose depot volume information, or vascularization bed dimension information. In an embodiment, a computing device 114 actuates an alignment of plurality of treatment focal regions 104 with the detected one or more adipose depot targets 106 based on the real-time detected adipose depot information.

In an embodiment, the apoptosis inducement module 150 is configured to update a user-specific treatment protocol in response to the delivery of the pro-apoptotic energy stimulus 103. In an embodiment, the apoptosis inducement module 150 is configured to update a user-specific treatment protocol based on one or more spectral components associated with the at least one measurand.

In an embodiment, the target apoptosis induction module 150 includes one or more memories 134 configured to store at least one of target-specific treatment information, user-specific treatment history, or previous-in-time treatment history. In an embodiment, the apoptosis induction module 150 is configured to determine a treatment protocol of pro-apoptotic energy based on previous-in-time treatment history. In an embodiment, the apoptosis induction module 150 is configured to determine a multi-session treatment protocol of pro-apoptotic energy delivery based on previous-in-time treatment history. In an embodiment, the apoptosis induction module 150 is configured to determine a treatment protocol associated with depleting adipose tissue within a target treatment region.

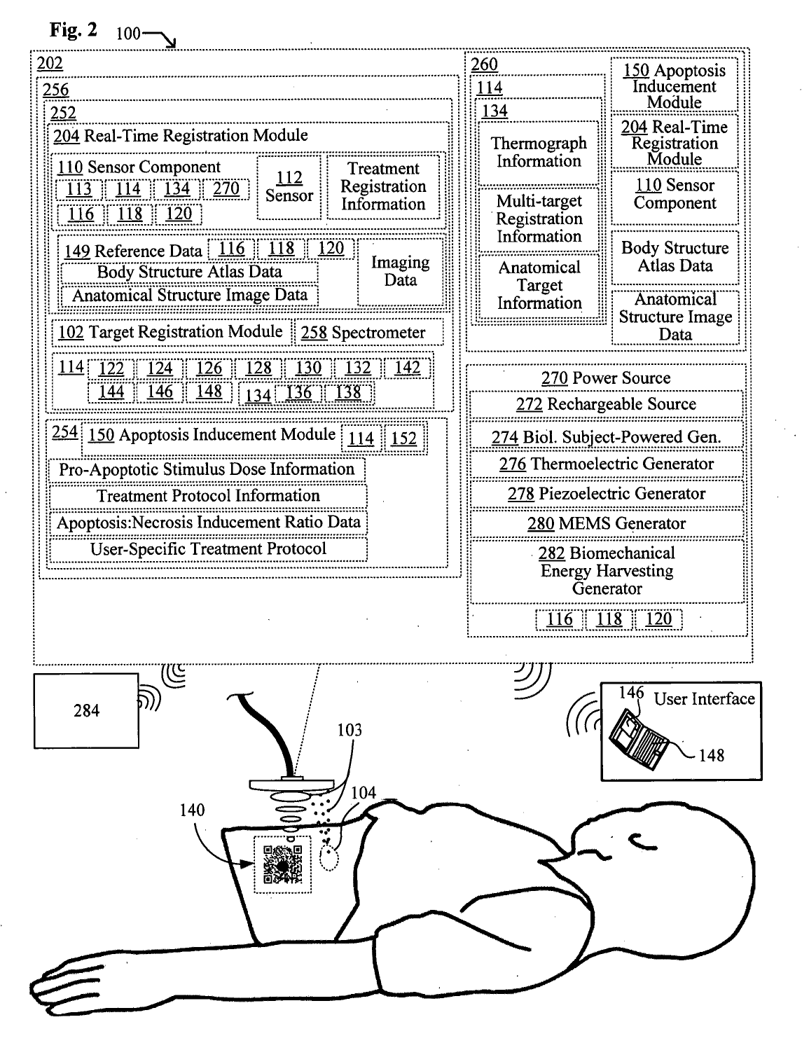

FIG. 2 shows a transcutaneous energy delivery apparatus 202 in which one or more methodologies or technologies can be implemented such as, for example, inducing programmed cell death within one or more treatment focal regions, or the like. In an embodiment, the transcutaneous energy delivery apparatus 202 includes a real-time registration module 204 configured to register one or more treatment focal regions 104 with at least one anatomical target and to generate treatment registration information. Non-limiting examples of treatment registration information include one or more of a treatment focal region location coordinate, a treatment focal region dimension, a treatment focal region depth, or a treatment focal region beam axis direction. Further non-limiting examples of treatment registration information include one or more of identification, location, shape, dimension, or distribution. In an embodiment, the registration information includes a point cloud associated with at least one of an anatomical target, a non-treatment region, a biological structure, a subsurface anatomical structure, etc.

In an embodiment, the real-time registration module 204 includes at least one sensor component 110 configured to acquire a thermograph of the at least one anatomical target at one or more fields of view. In an embodiment, one or more computing devices 114 are configured to determine a spatial frequency spectrum of at least a first subset of pixels of a thermograph, and to generate treatment registration information based on a comparison of the spatial frequency spectrum of the first subset of pixels to reference thermograph information. In an embodiment, the real-time registration module 204 includes at least one sensor component 110 configured to determine amount of adipose tissue within the one or more treatment focal regions. For example, in an embodiment, the real-time registration module 204 includes at least spectrometer 113 configured to determine amount of adipose tissue within the one or more treatment focal regions.

In an embodiment, the real-time registration module 204 includes at least one sensor component 110 configured to estimate amount of adipose cells within the at least one anatomical target. In an embodiment, the real-time registration module 204 includes at least one sensor component 110 configured to estimate an amount of adipose cells within the at least one anatomical target based on a response to an energy interrogation stimulus.

In an embodiment, the real-time registration module 204 includes a sensor component 110 configured to detect a location of a peripheral vascular bed. In an embodiment, the real-time registration module 204 registers the one or more treatment focal regions 104 with the at least one anatomical target based on the detected location of the peripheral vascular bed. In an embodiment, the real-time registration module 204 includes a sensor component 110 configured to detect and track a location of peripheral vascular beds relative to the movement of the transcutaneous energy delivery apparatus.

In an embodiment, the real-time registration module 204 includes one or more computing devices 114 configured to compare a real-time detected measurand associated with one or more subsurface anatomical targets to reference subsurface anatomical target information, and to generate treatment registration information based on the comparison. In an embodiment, the real-time registration module 204 includes a sensor component 110 operably coupled to a computing device 114 that actuates an alignment of the one or more treatment focal regions 104 with one or more anatomical targets based on a detected measurand associated with a biological structure.

In an embodiment, the real-time registration module 204 includes a sensor component 110 configured to detect and track a relative spacing between the at least one anatomical target and the one or more treatment focal regions 104. In an embodiment, the sensor component 110 is configured to real-time motion-track the at least one anatomical target and the one or more treatment focal regions 104. For example, in an embodiment, the real-time registration module 204 includes one or more inertial sensors that motion-track at least one of a position, orientation, or movement of the transcutaneous energy delivery apparatus. In an embodiment, the sensor component 110 is configured to motion-track the movement of the at least one anatomical target relative to the one or more treatment focal regions 104.

In an embodiment, the real-time registration module 204 is configured to register one or more treatment focal regions and an anatomical target relative to a coordinate reference associated with the transcutaneous energy delivery apparatus 202. In an embodiment, the real-time registration module 204 includes a sensor component 110 configured to detect one or more surface markings 140. In an embodiment, the detection of one or more surface markings 140 informs the real-time registration module 204 regarding a coordinate reference frame to aid in registering one or more treatment focal regions 104 with one or more anatomical target. For example, in an embodiment, during operation, the target registration module 102 registers one or more treatment focal regions 104 with one or more anatomical targets relative to the detected one or more surface markings 140.

In an embodiment, the real-time registration module 204 is configured to generate a treatment protocol based on the treatment registration information. In an embodiment, the real-time registration module 204 is configured to register a location of a plurality of treatment focal regions 104 and to generate at least a first-in-time treatment protocol and a second-in-time treatment protocol based on the treatment registration information. In an embodiment, the real-time registration module 204 is configured to motion-track the movement of the at least one anatomical target, and real-time position the one or more treatment focal regions 104 onto the at least one anatomical target. For example, in an embodiment, the real-time registration module 204 includes a sensor component 110 configured to determine a location of the at least one anatomical target by monitoring a metabolic process.

In an embodiment, the real-time registration module 204 is configured to detect and track anatomical targets and to synchronize treatment delivery to the one or more treatment focal regions 104 with a motion of the anatomical targets. In an embodiment, the real-time registration module 204 is configured to register the one or more treatment focal regions 104 with one or more surface markings 140 and to generate treatment registration information. In an embodiment, the real-time registration module 204 is configured to register the one or more treatment focal regions 104 with one or more surface markings 140 and to generate treatment registration information.

In an embodiment, the real-time registration module 204 locates an anatomical target using at least one of an artificial body surface marking, a tattoo, or a plurality of nanoparticle fiducial markers, and registers one or more treatment focal regions 104 with the anatomical target located using at least one of an artificial body surface marking, a tattoo, or a plurality of nanoparticle fiducial markers and to generate treatment registration information. In an embodiment, the real-time registration module 204 locates an anatomical target for registration using at least one of computerized axial tomography, fiber optic thermometry, infrared thermography, magnetic resonance imaging, magnetic resonance spectroscopy, microwave thermography, microwave dielectric spectroscopy, positron emission tomography, ultrasound reflectometry, spectroscopic imaging, visual imaging, infrared imaging, or single photon emission computed tomography.

In an embodiment, the real-time registration module 204 is configured to detect and track subsurface anatomical targets using subsurface thermography. For example, in an embodiment, the real-time registration module 204 includes one or more computing devices 114 operably coupled to a thermal imaging system. In an embodiment, the one or more computing devices 114 compare a detected thermograph obtained by the thermal imaging system to reference thermograph information, and generate treatment registration information based on the comparison. In an embodiment, the real-time registration module 204 includes one or more computing devices 114 configured to compare a detected dielectric profile associated with one or more subsurface anatomical targets to reference dielectric information and to generate treatment registration information based on the comparison. In an embodiment, the real-time registration module 204 is configured to identify groups of pixels in a thermograph indicative of at least one anatomical target imaged in the thermograph, and to generate treatment registration information representative of a parameter associated with a location and a dimension of the one or more treatment focal regions 104.

In an embodiment, the real-time registration module 204 is configured to register at least one non-treatment target and to generate non-treatment registration information. For example, in an embodiment, the real-time registration module 204 registers one or more non-treatment targets and one or more adipose depot targets 106 and generates treatment and non-treatment registration information. In an embodiment, the real-time registration module 204 registers one or more brown adipose depots as non-treatment targets and one or more white adipose depots as treatment targets and generates treatment and non-treatment registration information.

In an embodiment, the transcutaneous energy delivery apparatus 202 includes an apoptosis induction module 150 configured to transcutaneously deliver a pro-apoptotic energy stimulus 103 to the one or more treatment focal regions 104 based. on the treatment registration information. In an embodiment, the apoptosis induction module 150 includes one or more energy emitters 152 configured to transcutaneously deliver a multi-focal pro-apoptotic energy stimulus 103. In an embodiment, the apoptosis induction module 150 includes one or more energy emitters 152 configured to transcutaneously deliver the pro-apoptotic energy stimulus 103 at a dose sufficient to induce programmed cell death, without substantially inducing necrosis, of adipocytes within the one or more treatment focal regions 104. In an embodiment, the apoptosis induction module 150 includes one or more energy emitters 152 configured to transcutaneously deliver the pro-apoptotic energy stimulus 103 at a dose sufficient to induce apoptosis in a target amount of adipocytes within the one or more treatment focal regions 104. In an embodiment, the apoptosis induction module 150 includes one or more energy emitters 152 configured to transcutaneously deliver the pro-apoptotic energy stimulus 103 having frequency, power, duration, intensity, duty cycle, or pulse rate sufficient to induce apoptosis in a target amount of adipocytes.

In an embodiment, the transcutaneous energy delivery apparatus 202 includes one or more energy emitters 152 configured to transcutaneously deliver the pro-apoptotic energy stimulus 103 at a dose sufficient to induce programmed cell death of adipose tissue within the one or more treatment focal regions 104, without substantially inducing programmed cell death of non-adipose tissue. In an embodiment, the transcutaneous energy delivery apparatus 202 includes one or more energy emitters 152 configured to transcutaneously deliver the pro-apoptotic energy stimulus 103 at a dose sufficient to induce programmed cell death of adipocytes within the one or more treatment focal regions 104, without substantially inducing programmed cell death of tissue proximate the one or more treatment focal regions 104.

In an embodiment, the transcutaneous energy delivery apparatus 202 includes one or more energy emitters 152 configured to transcutaneously deliver the pro-apoptotic energy stimulus 103 at a dose sufficient to induce programmed cell death of adipocytes within the one or more treatment focal regions, without substantially allowing treatment focal regions 104 to induce programmed cell death of overlying tissue. In an embodiment, the transcutaneous energy delivery apparatus 202 includes one or more energy emitters 152 configured to transcutaneously deliver at least one of a pro-apoptotic electromagnetic energy stimulus, a pro-apoptotic electrical energy stimulus, a pro-apoptotic acoustic energy stimulus, or a pro-apoptotic thermal energy stimulus.

In an embodiment, the transcutaneous energy delivery apparatus 202 includes one or more energy emitters 152 that concurrently or sequentially deliver one or more of a pro-apoptotic electromagnetic energy stimulus, a pro-apoptotic electrical energy stimulus, a pro-apoptotic acoustic energy stimulus, or a pro-apoptotic thermal energy stimulus. In an embodiment, the apoptosis induction module 150 includes a plurality of energy emitters 152 that concurrently or sequentially deliver one or more of a focused ultrasound energy stimulus, a focused electromagnetic energy stimulus, or a focused microwave stimulus.

In an embodiment, the transcutaneous energy delivery apparatus 202 includes at least one radiofrequency phased array configured to transcutaneously deliver a focused microwave stimulus at a dose sufficient to induce programmed cell death of adipocytes within the one or more treatment focal regions 104, without substantially inducing programmed cell death of non-adipocytes. In an embodiment, the apoptosis induction module 150 includes a transducer array configured to transcutaneously deliver a focused ultrasound energy stimulus at a dose sufficient to induce programmed cell death of adipocytes within the one or more treatment focal regions 104, without substantially inducing programmed cell death of non-adipocytes.

In an embodiment, the transcutaneous energy delivery apparatus 202 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 3° C. to about 22° C., without substantially elevating a temperature of overlying tissue. In an embodiment, the transcutaneous energy delivery apparatus 202 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 3° C. to about 10° C., without substantially elevating a temperature of overlying tissue. In an embodiment, the transcutaneous energy delivery apparatus 202 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 3° C. to about 4° C., without substantially elevating a temperature of overlying tissue.

In an embodiment, the transcutaneous energy delivery apparatus 202 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 37° C. to less than about 60° C., without substantially elevating a temperature of overlying tissue. In an embodiment, the transcutaneous energy delivery apparatus 202 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 37° C. to less than about 47° C., without substantially elevating a temperature of overlying tissue. In an embodiment, the transcutaneous energy delivery apparatus 202 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 37° C. to less than about 45° C., without substantially elevating a temperature of overlying tissue. In an embodiment, the transcutaneous energy delivery apparatus 202 includes one or more energy emitters 152 configured to transcutaneously deliver a pro-apoptotic energy stimulus 103 at a dose sufficient to elevate a temperature of at least a portion of adipocytes within the one or more treatment focal regions 104 from about 37° C. to less than about 42° C., without substantially elevating a temperature of overlying tissue. In an embodiment, the transcutaneous energy delivery apparatus 202 includes one or more energy emitters 152 configured to transcutaneously deliver a pro-apoptotic energy stimulus 103 at a dose sufficient to elevate a temperature of at least a portion of adipocytes within the one or more treatment focal regions 104 from greater than about 41° C. to less than about 63° C., without substantially elevating a temperature of overlying tissue.

In an embodiment, the transcutaneous energy delivery apparatus 202 includes an apoptosis induction module 150 having a memory 134 configured to store treatment registration information associated with the delivery of the pro-apoptotic energy stimulus 103 to the one or more treatment focal regions 104. In an embodiment, the transcutaneous energy delivery apparatus 202 includes an apoptosis induction module 150 configured to generate and store a next-in-time treatment protocol based on a comparison of the treatment registration information to patient specific treatment registration information.

In an embodiment, the transcutaneous energy delivery apparatus 202 includes at least one of a transceiver 120 or a receiver 116 configured to acquire (e.g., electromagnetically acquire, magnetically acquire, ultrasonically acquire , optically acquire, inductively acquire, electrically acquire, capacitively acquire, wirelessly acquire, or the like) treatment registration information. In an embodiment, the transcutaneous energy delivery apparatus 202 includes at least one of a transceiver 120 or a receiver 116 configured to acquire at least one of previous-in-time treatment information or next-in-time treatment registration information. In an embodiment, the transcutaneous energy delivery apparatus 202 includes at least one of a transceiver 120 or a receiver 116 configured to receive a request to transmit treatment registration information.

In an embodiment, the transcutaneous energy delivery apparatus 202 includes at least one of a transceiver 120 or a receiver 116 configured to receive treatment protocol information. In an embodiment, the transcutaneous energy delivery apparatus 202 includes at least one of a transceiver 120 or a receiver 116 configured to receive an instruction to initiate transcutaneous delivery of the pro-apoptotic energy stimulus 103 to the one or more treatment focal regions 104. In an embodiment, the transcutaneous energy delivery apparatus 202 includes at least one of a transceiver 120 or a transmitter 118 configured to send treatment registration information. In an embodiment, the transcutaneous energy delivery apparatus 202 includes at least one of a transceiver 120 or a transmitter 118 configured to report a user status change.in response to the transcutaneous delivery of the pro-apoptotic energy stimulus 103.

In an embodiment, the transcutaneous energy delivery apparatus 202 includes a physical coupling element configured to removably-attach the transcutaneous energy delivery apparatus 202 to a biological surface of a biological subject. In an embodiment, at least one of the real-time registration module 204 or the apoptosis induction module 150 is configured for removable attachment to a biological surface of a biological subject. In an embodiment, at least one of the real-time registration module 204 or the apoptosis induction module 150 is sized and configured to be hand-held. In an embodiment, the apoptosis induction module 150 forms part of a hand-held pro-apoptotic energy stimulus 103 delivery component.

In an embodiment, the transcutaneous energy delivery apparatus 202 includes a target registration means 252. In an embodiment, the transcutaneous energy delivery apparatus 202 includes a target registration means 252 for aligning a treatment focal region 104 with an adipose depot target 106 and for generating treatment protocol information. In an embodiment, the target registration means 252 includes a sensor component 110 operably coupled to a computing device 114, the sensor component 110 configured to detect the adipose depot target 106 and the computing device 114 configured to cause the generation of treatment protocol information based on a registration of the treatment focal region 104 with a detected adipose depot target 106. For example, in an embodiment, the target registration means 252 includes a spectrometer 258 operably coupled to a computing device 114. In an embodiment, the computing device 114 generates real-time detected adipose depot information based on an output from the spectrometer 258 indicative of a location of an adipose depot target 106. In an embodiment, the target registration means 252 includes a memory 134 operably coupled to a computing device 114 configured to actuate an alignment of the at least one treatment focal region 104 with a detected adipose depot target 106 based on real-time detected adipose depot information.

In an embodiment, the transcutaneous energy delivery apparatus 202 includes an apoptosis induction means 254. In an embodiment, the transcutaneous energy delivery apparatus 202 includes an apoptosis induction means 254 for transcutaneously delivering an energy stimulus to at least one treatment focal region 104. In an embodiment, the apoptosis induction means 254 includes a computing device 114 operably coupled to a plurality of energy emitters 152. During operation, in an embodiment, the computing device 114 actuates the transcutaneous delivery of an electromagnetic stimulus to the at least one treatment focal region 104 by one or more of a plurality of energy emitters 152 based on the treatment protocol information. In an embodiment, the apoptosis induction means 254 includes a computing device 114 operably coupled to a plurality of transducers, and configured to actuate the transcutaneous delivery of a focused ultrasound stimulus to the treatment focal region 104 by one or more of the plurality of transducers based on the treatment protocol information. In an embodiment, the apoptosis induction means 254 includes a computing device 114 operably coupled to a memory 134, and is configured to cause a storing of treatment protocol information at a plurality of time intervals.

In an embodiment, the transcutaneous energy delivery apparatus 202 includes a target tracking means 256 including a sensor component 110 and a computing device operably coupled to the sensor component 110 and the apoptosis induction means 254. In an embodiment, the target tracking means 256 registers a treatment focal region location within the body of a biological subject relative to a reference location and to alter a duty cycle associated with the transcutaneous delivery of the energy stimulus based on the registering of the treatment focal region location within the body relative to the reference location.

In an embodiment, the transcutaneous energy delivery apparatus 202 includes a target identification and registration module 260 configured to identify a treatment target of a biological subject based on a detected measurand, and to align a plurality of treatment focal regions with the treatment target. In an embodiment, the detected measurand includes a temperature, an electrical resistivity, an electrical conductivity, a magnetic susceptibility, an elasticity, or a density. In an embodiment, the detected measurand includes a measurand associated with computerized axial tomography, fiber optic thermometry, infrared thermography, magnetic resonance imaging, magnetic resonance spectroscopy, microwave thermography, microwave dielectric spectroscopy, positron emission tomography, ultrasound reflectometry, spectroscopic imaging, visual imaging, infrared imaging, or single photon emission computed tomography.

In an embodiment, the target identification and registration module 260 generates treatment registration information indicative of an alignment of a plurality of treatment focal regions 104 with a treatment target based on a reference coordinate frame associated with an anatomical feature of a user. In an embodiment, the target identification and registration module 260 is configured to generate at least one of an adipose depot location, an adipose depot composition, or an adipose depot volume. In an embodiment, the target identification and registration module 260 is configured to generate at least one of an adipose depot thermal profile or an adipose depot dielectric profile. In an embodiment, the target identification and registration module 260 is configured to determine the dose of the pro-apoptotic energy based on a probability of inducing apoptosis of at least a portion of the treatment target, a probability of inducing necrosis of at least a portion of the treatment target, or a combination thereof.

In an embodiment, the target identification and registration module 260 determine the dose of the pro-apoptotic energy based on an estimated apoptosis:necrosis inducement ratio. In an embodiment, the estimated apoptosis:necrosis inducement ratio is based on previous-in-time treatment . information. In an embodiment, the estimated apoptosis:necrosis inducement ratio is determined using a real-time measurand associated with the one or more adipose depot targets 106 treated with the pro-apoptotic energy stimulus 103. In an embodiment, the target identification and registration module 260 determines the dose of the pro-apoptotic energy based on a probability of inducing programmed cell death, without substantially inducing necrosis, of adipocytes within the treatment target. In an embodiment, the target identification and registration module 260 is configured to determine the dose of the pro-apoptotic energy based on an apoptosis: necrosis inducement ratio. In an embodiment, the target identification and registration module 260 is configured to determine the dose of the pro-apoptotic energy based on probability of inducing apoptosis of adipocytes within the treatment target and to determine a confidence level associated with the probability of inducing apoptosis of adipocytes within the treatment target. In an embodiment, the target identification and registration module 260 is configured to store user-specific treatment history.

In an embodiment, the system 100 includes a transcutaneous energy delivery apparatus 202 having a target registration module 102 that indicates, via one or more of a visual, audio, haptic, or a tactile representation, that the one or more treatment focal regions 104 are in a location position to deliver treatment. For example, in an embodiment, the system 100 instructs a user, via one or more of a visual, audio, haptic, or a tactile representation, on how to move or where to position a transcutaneous energy delivery apparatus 202 for treatment delivery (e.g., two inches to the left, move a given distance in the direction indicated on a display, etc.). In an embodiment, the transcutaneous energy delivery apparatus 202 toggles between at least a first configuration operable to deliver a pro-apoptotic energy stimulus 103 and a second configuration operable to deactivate, cease, etc., energy delivery based on whether registration indicates that the treatment focal region 104 coincides with a treatment target. For example, during operation, the target registration module 102 motion tracks one or more treatment targets, generates an indication that the one or more treatment focal regions 104 are in the proper location to deliver treatment, and actuates the first configuration operable to deliver a pro-apoptotic energy stimulus 103. Conversely, in an embodiment, the transcutaneous energy delivery apparatus 202 ceases energy delivery when registration indicates that the treatment focal region 104 is not in registration with a treatment target (or is in registration with a non-treatment target). In an embodiment, the transcutaneous energy delivery apparatus 202 actuates a non-treatment delivery configuration when out of position (e.g., when the target registration information indicates that the treatment focal region 104 is not in proper registration with an anatomical target, etc.). In an embodiment, the transcutaneous energy delivery apparatus 202 actuates a treatment delivery configuration when in position (e.g., when the target registration information indicates that the treatment focal region 104 is in proper registration with an anatomical target, etc.).

In an embodiment, the system 100 includes, among other things, one or more power sources 270. In an embodiment, the transcutaneous energy delivery apparatus 202 includes one or more power sources 270. In an embodiment, the power source 270 is electromagnetically, magnetically, acoustically, optically, inductively, electrically, or capacitively coupled to at least one of the target registration module 102, the apoptosis inducement module 150, the target identification and registration module 260, a sensor component 110, a computing device 114, an energy emitter 152, or the like. Non-limiting examples of power sources 270 examples include one or more button cells, chemical battery cells, a fuel cell, secondary cells, lithium ion cells, micro-electric patches, nickel metal hydride cells, silver-zinc cells, capacitors, super-capacitors, thin film secondary cells, ultra-capacitors, zinc-air cells, or the like. Further non-limiting examples of power sources 270 include one or more generators (e.g., electrical generators, thermo energy-to-electrical energy generators, mechanical-energy-to-electrical energy generators, micro-generators, nano-generators, or the like) such as, for example, thermoelectric generators, piezoelectric generators, electromechanical generators, biomechanical-energy harvesting generators, or the like. In an embodiment, the power source 270 includes at least one rechargeable power source 272. In an embodiment, the transcutaneous energy delivery apparatus 202 carries the power source 270. In an embodiment, the transcutaneous energy delivery apparatus 202 includes at least one of a battery, a capacitor, or a mechanical energy store (e.g., a spring, a flywheel, or the like).

In an embodiment, the power source 270 is configured to manage a duty cycle associated with a transcutaneous delivery of the pro-apoptotic energy. For example, in an embodiment, the power source 270 is configured to manage a duty cycle based on at least one of a comparison of a detected measurand to a user-specific treatment protocol; a time variable behavior of a relative movement between the plurality of treatment focal regions 104 and the treatment target; or the like. In an embodiment, the transcutaneous energy delivery apparatus 202 is configured to provide a voltage, via a power source 270 operably coupled to at least one of the target registration module 102, the apoptosis inducement module 150, or the target identification and registration module 260.

In an embodiment, the power source 270 is configured to wirelessly receive power from a remote power supply. For example, in an embodiment, the power source 270 receives power from a remote power supply via one or more transceivers 120 or receivers 116. In an embodiment, the transcutaneous energy delivery apparatus 202 includes one or more power receivers configured to receive power from an in vivo or ex vivo power source. In an embodiment, the power source 270 is configured to wirelessly receive power via at least one of an electrical conductor or an electromagnetic waveguide. In an embodiment, the power source 270 includes one or more power receivers configured to receive power from an in vivo or ex vivo power source. In an embodiment, the in vivo power source includes at least one of a thermoelectric generator, a piezoelectric generator, a microelectromechanical systems generator, or a biomechanical-energy harvesting generator.

In an embodiment, the transcutaneous energy delivery apparatus 202 includes one or more generators configured to harvest mechanical energy from, for example, acoustic waves, mechanical vibration, blood flow, or the like. For example, in an embodiment, the power source 270 includes at least one of a biological-subject (e.g., human)-powered generator 274, a thermoelectric generator 276, a piezoelectric generator 278, an electromechanical generator (e.g., a microelectromechanical systems (MEMS) generator 280, or the like), a biomechanical-energy harvesting generator 282, or the like.

In an embodiment, the biological-subject-powered generator 274 is configured to harvest thermal energy generated by the biological subject. In an embodiment, the biological-subject-powered generator 274 is configured to harvest energy generated by the biological subject using at least one of a thermoelectric generator 276, a piezoelectric generator 278, an electromechanical generator 280 (e.g., a microelectromechanical systems (MEMS) generator, or the like), a biomechanical-energy harvesting generator 282, or the like. For example, in an embodiment, the biological-subject-powered generator 274 includes one or more thermoelectric generators 276 configured to convert heat dissipated by the biological subject into electricity. In an embodiment, the biological-subject-powered generator 274 is configured to harvest energy generated by any physical motion or movement (e.g., walking,) by biological subject. For example, in an embodiment, the biological-subject-powered generator 274 is configured to harvest energy generated by the movement of a joint within the biological subject. In an embodiment, the biological-subject-powered generator 274 is configured to harvest energy generated by the movement of a fluid (e.g., biological fluid, etc.) within the biological subject.

In an embodiment, the system 100 includes, among other things, a transcutaneous energy transfer system 284. In an embodiment, the transcutaneous energy delivery apparatus 202 includes a transcutaneous energy transfer system 284. For example, in an embodiment, the transcutaneous energy delivery apparatus 202 includes one or more power receivers configured to receive power from at least one of an in vivo power source. In an embodiment, the transcutaneous energy transfer system 284 is electromagnetically, magnetically, acoustically, optically, inductively, electrically, or capacitively coupleable to an in vivo power supply. In an embodiment, the transcutaneous energy transfer system 284 includes at least one electromagnetically coupleable power supply, magnetically coupleable power supply, acoustically coupleable power supply, optically coupleable power supply, inductively coupleable power supply, electrically coupleable power supply, or capacitively coupleable power supply. In an embodiment, the energy transcutaneous transfer system is configured to wirelessly receive power from a remote power supply.

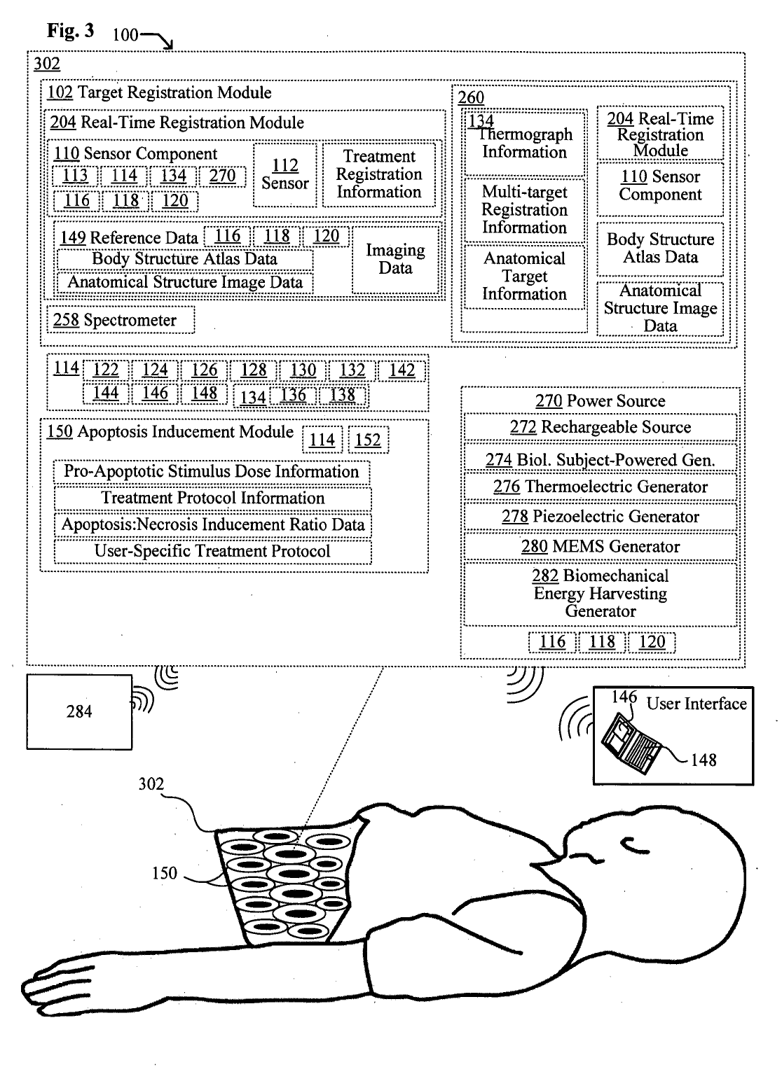

FIG. 3 shows an energy delivery apparatus 302 in which one or more methodologies or technologies can be implemented such as, for example, inducing programmed cell death, without substantially inducing necrosis, of adipose depot target 106 within one or more treatment focal regions 104, or the like. In an embodiment, the energy delivery apparatus 302 includes a target registration module 102 configured to align at least one treatment focal region 104 with one or more adipose depot targets 106.

In an embodiment, the energy delivery apparatus 302 includes an apoptosis inducement module 150 configured to deliver a pro-apoptotic energy stimulus 103 to the at least one treatment focal region 104 according to a treatment cycle based on an estimated apoptosis:necrosis inducement ratio. In an embodiment, the treatment cycle is based on a probability of inducing apoptosis of one or more depot targets within the at least one treatment focal region, a probability of inducing necrosis of one or more depot targets within the at least one treatment focal region, or a combination thereof.

In an embodiment, the apoptosis inducement module 150 is configured to alter the treatment cycle associated with a delivery of the pro-apoptotic energy stimulus 103 to the at least one treatment focal region 104 based on the estimated apoptosis:necrosis inducement ratio. In an embodiment, the apoptosis inducement module 150 is configured to alter the treatment cycle associated with a delivery of the pro-apoptotic energy stimulus 103 to the at least one treatment focal region 104 based on a probability of inducing apoptosis of one or more depot targets within the at least one treatment focal region, a probability of inducing necrosis of one or more depot targets within the at least one treatment focal region, or a combination thereof. In an embodiment, the target registration module 102 registers the at least one treatment focal region 104 with the one or more adipose depot targets 106 using at least one of computerized axial tomography, fiber optic thermometry, infrared thermography, magnetic resonance imaging, magnetic resonance spectroscopy, microwave thermography, microwave dielectric spectroscopy, positron emission tomography, ultrasound reflectometry, spectroscopic imaging, visual imaging, infrared imaging, or single photon emission.

In an embodiment, the target registration module 102 registers the at least one treatment focal region 104 with the one or more adipose depot targets 106 using peripheral vascularization location information. In an embodiment, the target registration module 102 registers the at least one treatment focal region 104 with the one or more adipose depot targets 106 using reference multi-target registration information. In an embodiment, the target registration module 102 is configured to register a cumulative motion of the energy delivery apparatus relative to the one or more adipose depot targets 106 and to align the at least one treatment focal region 104 with the one or more adipose depot targets 106 using motion tracking.

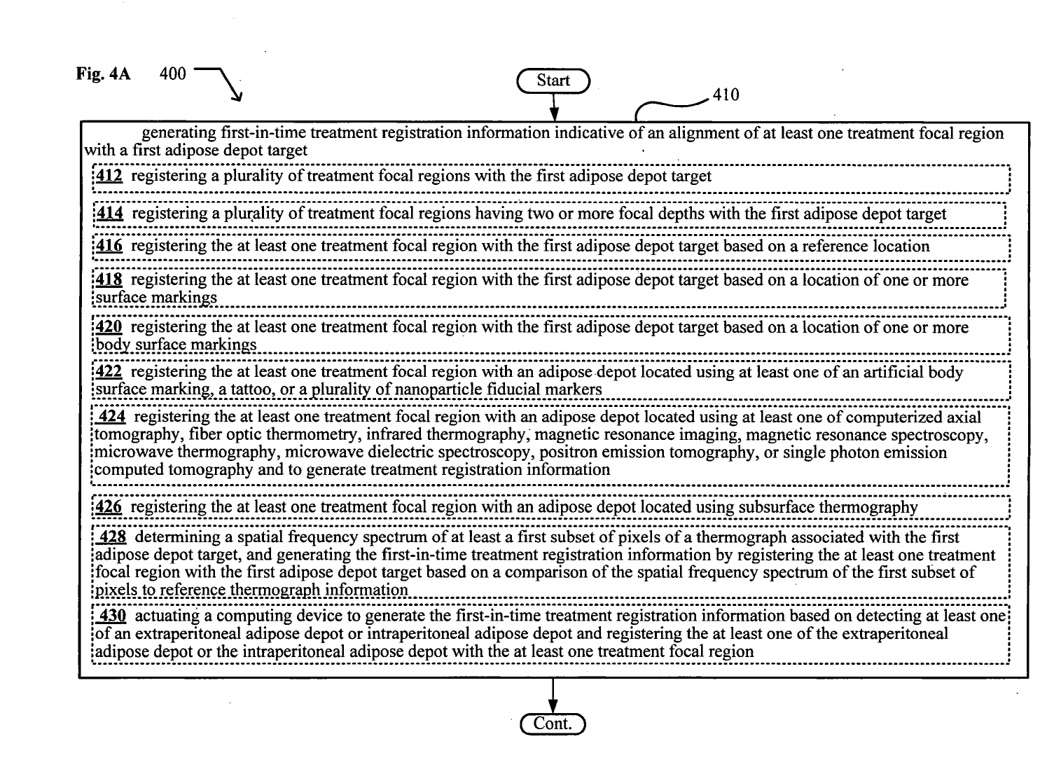

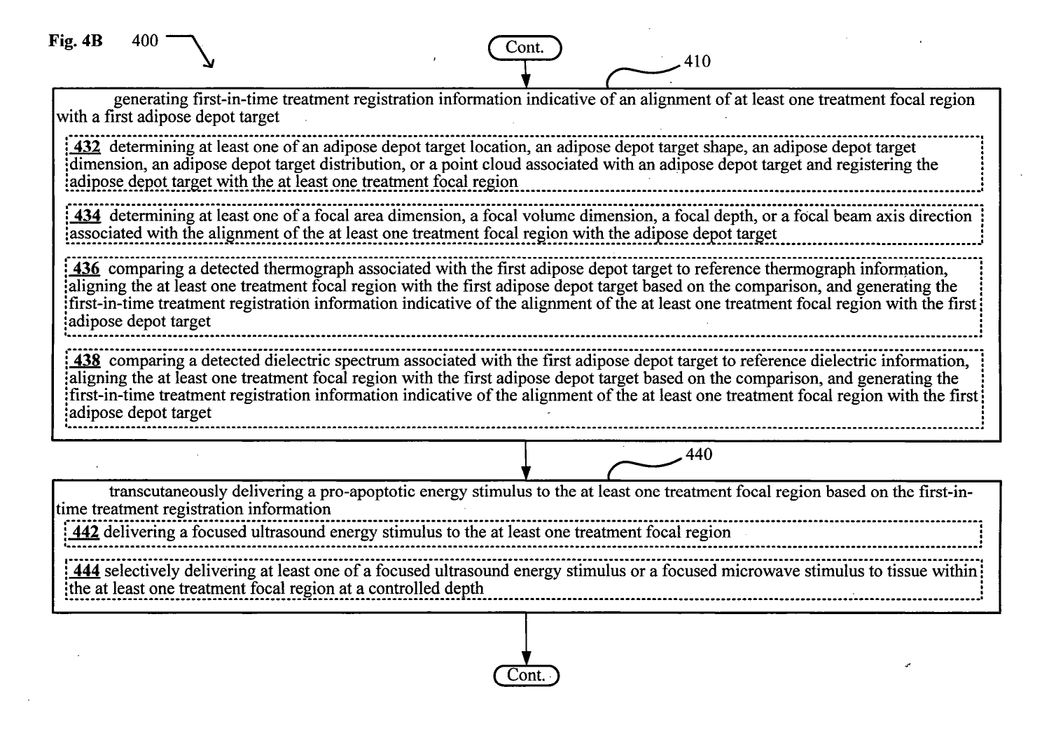

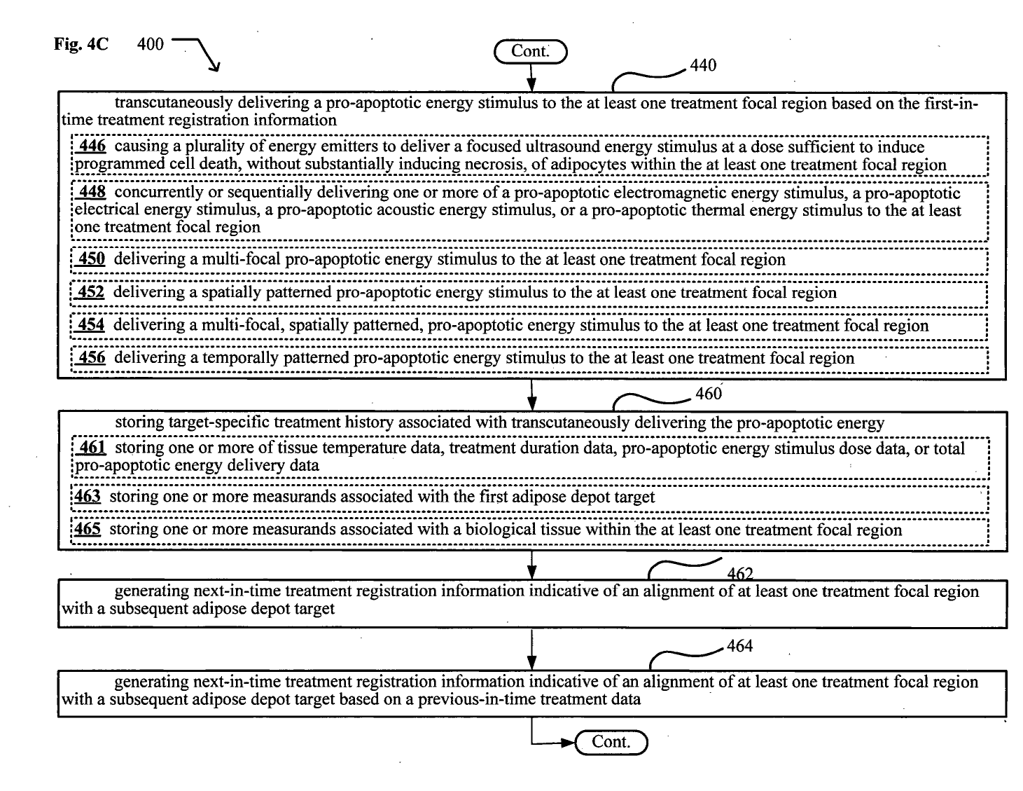

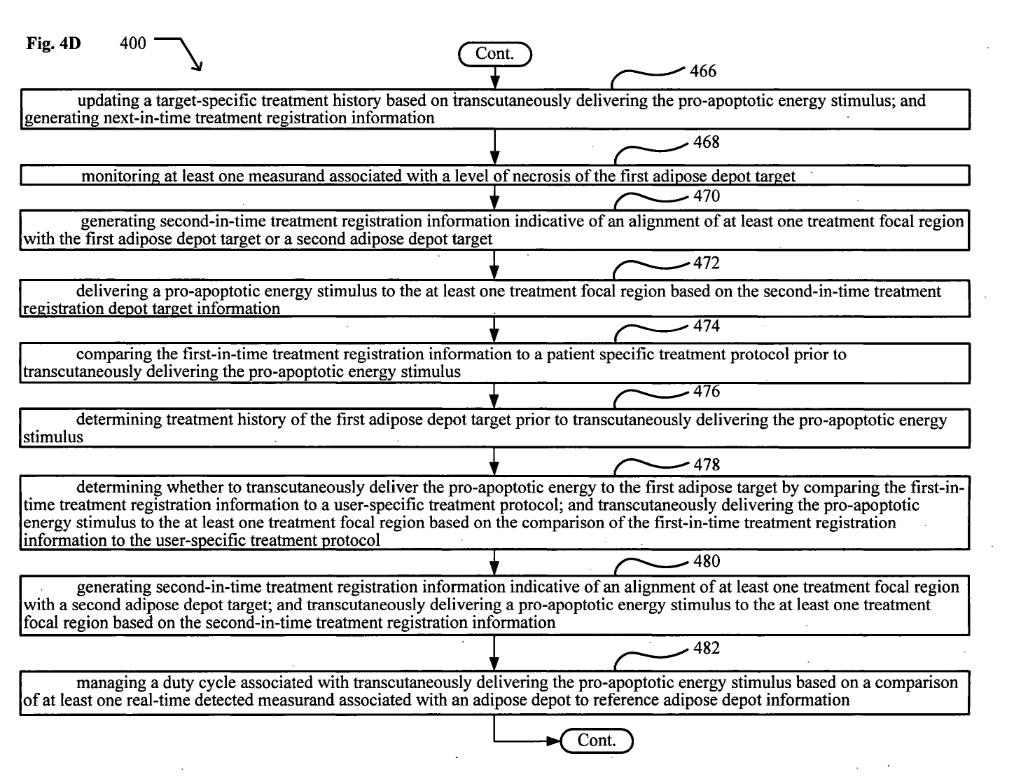

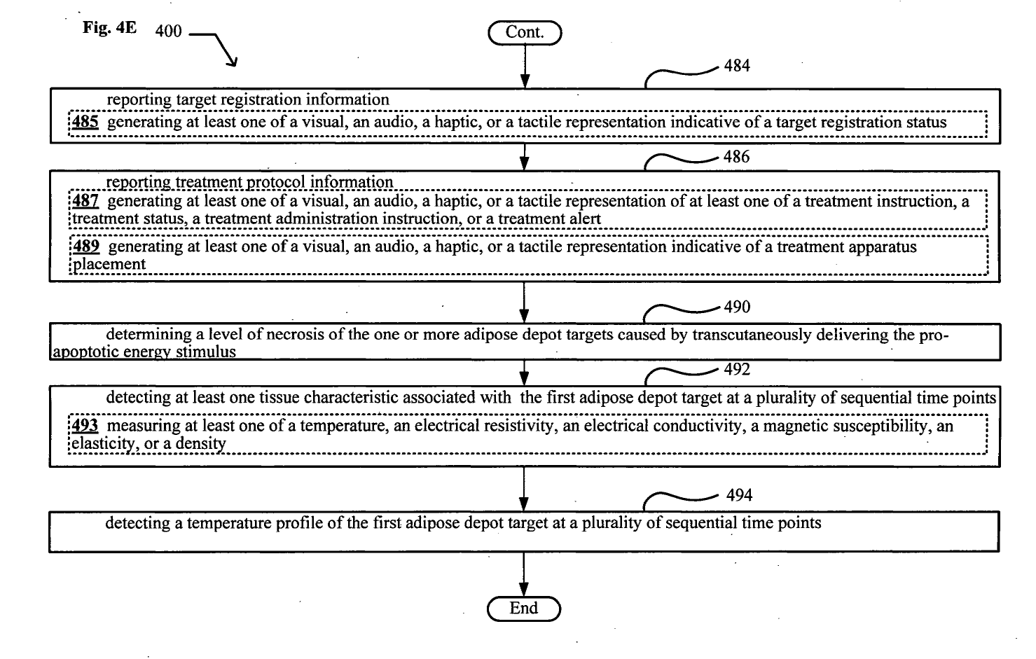

FIGS. 4A, 4B, 4C, 4D and 4E show a method 400. At 410, the method 400 includes generating first-in-time treatment registration information indicative of an alignment of at least one treatment focal region 104 with a first adipose depot target 106. At 412, generating the first-in-time treatment registration information includes registering a plurality of treatment focal regions 104 with the first adipose depot target. At 414, generating the first-in-time treatment registration information includes registering a plurality of treatment focal regions 104 having two or more focal depths with the first adipose depot target. At 416, generating the first-in-time treatment registration information includes registering the at least one treatment focal region 104 with the first adipose depot target based on a reference location.

At 418, generating the first-in-time treatment registration information includes registering the at least one treatment focal region 104 with the first adipose depot target based on a location of one or more surface markings 140. At 420, generating the first-in-time treatment registration information includes registering the at least one treatment focal region 104 with the first adipose depot target based on a location of one or more surface markings 140. At 422, generating the first-in-time treatment registration information includes registering the at least one treatment focal region 104 with an adipose depot located using at least one of an artificial body surface marking, a tattoo, or a plurality of nanoparticle fiducial markers.

At 424, generating the first-in-time treatment registration information includes registering the at least one treatment focal region 104 with an adipose depot located using at least one of computerized axial tomography, fiber optic thermometry, infrared thermography, magnetic resonance imaging, magnetic resonance spectroscopy, microwave thermography, microwave dielectric spectroscopy, positron emission tomography, ultrasound reflectometry, spectroscopic imaging, visual imaging, infrared imaging, or single photon emission computed tomography and to generate treatment registration information. At 426, generating the first-in-time treatment registration information includes registering the at least one treatment focal region 104 with an adipose depot located using subsurface thermography.

At 428, generating the first-in-time treatment registration information includes determining a spatial frequency spectrum of at least a first subset of pixels of a thermograph associated with the first adipose depot target, and generating the first-in-time treatment registration information by registering the at least one treatment focal region 104 with the first adipose depot target based on a comparison of the spatial frequency spectrum of the first subset of pixels to reference thermograph information. At 430, generating the first-in-time treatment registration information includes actuating a computing device to generate the first-in-time treatment registration information based on detecting at least one of an extraperitoneal adipose depot or intraperitoneal adipose depot and registering the at least one of the extraperitoneal adipose depot or the intraperitoneal adipose depot with the at least one treatment focal region 104.

At 432, generating the first-in-time treatment registration information includes determining at least one of an adipose depot target location, an adipose depot target shape, an adipose depot target dimension, an adipose depot target 106 distribution, or a point cloud associated with an adipose depot target 106 and registering the adipose depot target 106 with the at least one treatment focal region 104. At 434, generating the first-in-time treatment registration information includes determining at least one of a focal area dimension, a focal volume dimension, a focal depth, or a focal beam axis direction associated with the alignment of the at least one treatment focal region 104 with the adipose depot target 106.

At 436, generating the first-in-time treatment registration information includes comparing a detected thermograph associated with the first adipose depot target to reference thermograph information, aligning the at least one treatment focal region 104 with the first adipose depot target based on the comparison, and generating the first-in-time treatment registration information indicative of the alignment of the at least one treatment focal region 104 with the first adipose depot target. At 438, generating the first-in-time treatment registration information includes comparing a detected dielectric spectrum associated with the first adipose depot target to reference dielectric information, aligning the at least one treatment focal region 104 with the first adipose depot target based on the comparison, and generating the first-in-time treatment registration information indicative of the alignment of the at least one treatment focal region 104 with the first adipose depot target.

At 440, the method 400 includes transcutaneously delivering a pro-apoptotic energy stimulus 103 to the at least one treatment focal region 104 based on the first-in-time treatment registration information. In an embodiment, transcutaneously delivering the pro-apoptotic energy stimulus 103 includes causing a plurality of energy emitters 152 to deliver an energy stimulus at a dose sufficient to induce programmed cell death of adipocytes within the at least one treatment focal region 104. At 442, transcutaneously delivering the pro-apoptotic energy stimulus 103 includes delivering a focused ultrasound energy stimulus to the at least one treatment focal region 104. At 444, transcutaneously delivering the pro-apoptotic energy stimulus 103 includes selectively delivering at least one of a focused ultrasound energy stimulus or a focused microwave stimulus to tissue within the at least one treatment focal region 104 at a controlled depth.

At 446, transcutaneously delivering the pro-apoptotic energy stimulus 103 includes causing a plurality of energy emitters 152 to deliver a focused ultrasound energy stimulus at a dose sufficient to induce programmed cell death, without substantially inducing necrosis, of adipocytes within the at least one treatment focal region 104. At 448, transcutaneously delivering the pro-apoptotic energy stimulus 103 includes concurrently or sequentially delivering one or more of a pro-apoptotic electromagnetic energy stimulus, a pro-apoptotic electrical energy stimulus, a pro-apoptotic acoustic energy stimulus, or a pro-apoptotic thermal energy stimulus to the at least one treatment focal region 104. At 450, transcutaneously delivering the pro-apoptotic energy stimulus 103 includes delivering a multi-focal pro-apoptotic energy stimulus to the at least one treatment focal region 104.

At 452, transcutaneously delivering the pro-apoptotic energy stimulus 103 includes delivering a spatially patterned pro-apoptotic energy stimulus 103 to the at least one treatment focal region 104. At 454, transcutaneously delivering the pro-apoptotic energy stimulus 103 includes delivering a multi-focal, spatially patterned, pro-apoptotic energy stimulus to the at least one treatment focal region 104. At 456, transcutaneously delivering the pro-apoptotic energy stimulus includes delivering a temporally patterned pro-apoptotic energy stimulus to the at least one treatment focal region 104.

At 460, the method 400 includes storing target-specific treatment history associated with transcutaneously delivering the pro-apoptotic energy. At 461, storing the target-specific treatment history includes storing one or more of tissue temperature data, treatment duration data, pro-apoptotic energy stimulus dose data, or total pro-apoptotic energy delivery data. At 463, storing the target-specific treatment history includes storing one or more measurands associated with the first adipose depot target. At 465, storing the target-specific treatment history includes storing one or more measurands associated with a biological tissue within the at least one treatment focal region.

At 462, the method 400 includes generating next-in-time treatment registration information indicative of an alignment of at least one treatment focal region 104 with a subsequent adipose depot target 106. At 464, the method 400 includes generating next-in-time treatment registration information indicative of an alignment of at least one treatment focal region 104 with a subsequent adipose depot target 106 based on a previous-in-time treatment data. At 466, the method 400 includes updating a target-specific treatment history based on transcutaneously delivering the pro-apoptotic energy stimulus 103; and generating next-in-time treatment registration information. At 468, the method 400 includes monitoring at least one measurand associated with a level of necrosis of the first adipose depot target. At 470, the method 400 includes generating second-in-time treatment registration information indicative of an alignment of at least one treatment focal region 104 with the first adipose depot target or a second adipose depot target. At 472, the method 400 includes delivering a pro-apoptotic energy stimulus 103 to the at least one treatment focal region 104 based on the second-in-time treatment registration depot target information.

At 474, the method 400 includes comparing the first-in-time treatment registration information to a patient specific treatment protocol prior to transcutaneously delivering the pro-apoptotic energy stimulus 103. At 476, the method 400 includes determining treatment history of the first adipose depot target prior to transcutaneously delivering the pro-apoptotic energy stimulus 103. At 478, the method 400 includes determining whether to transcutaneously deliver the pro-apoptotic energy to the first adipose target by comparing the first-in-time treatment registration information to a user-specific treatment protocol; and transcutaneously delivering the pro-apoptotic energy stimulus 103 to the at least one treatment focal region 104 based on the comparison of the first-in-time treatment registration information to the user-specific treatment protocol.

At 480, the method 400 includes generating second-in-time treatment registration information indicative of an alignment of at least one treatment focal region 104 with a second adipose depot target; and transcutaneously delivering a pro-apoptotic energy stimulus 103 to the at least one treatment focal region 104 based on the second-in-time treatment registration information. At 482, the method 400 includes managing a duty cycle associated with transcutaneously delivering the pro-apoptotic energy stimulus 103 based on a comparison of at least one real-time detected measurand associated with an adipose depot to reference adipose depot information. In an embodiment, the method 400 includes managing a duty cycle associated with transcutaneously delivering the pro-apoptotic energy stimulus 103 based on a comparison of a real-time detected adipose depot temperature to a reference temperature treatment protocol.

At 484, the method 400 includes reporting target registration information. At 485, reporting the target registration information includes generating at least one of a visual, an audio, a haptic, or a tactile representation indicative of a target registration status. In an embodiment, reporting the target registration information includes transmitting one or more of tissue temperature data, treatment duration data, pro-apoptotic energy stimulus dose data, or total pro-apoptotic energy delivery data. At 486, the method 400 includes reporting treatment protocol information. At 487, reporting the treatment protocol information includes generating at least one of a visual, an audio, a haptic, or a tactile representation of at least one of a treatment instruction, a treatment status, a treatment administration instruction, or a treatment alert. At 489, reporting the treatment protocol information includes generating at least one of a visual, an audio, a haptic, or a tactile representation indicative of a treatment apparatus placement.

At 490, the method 400 includes determining a level of necrosis of the one or more adipose depot targets caused by transcutaneously delivering the pro-apoptotic energy stimulus 103. At 492, the method 400 includes detecting at least one tissue characteristic associated with the first adipose depot target at a plurality of sequential time points. At 493, detecting the at least one tissue characteristic associated with the first adipose depot target includes measuring at least one of a temperature, an electrical resistivity, an electrical conductivity, a magnetic susceptibility, an elasticity, or a density. At 494, the method 400 includes detecting a temperature profile of the first adipose depot target at a plurality of sequential time points.

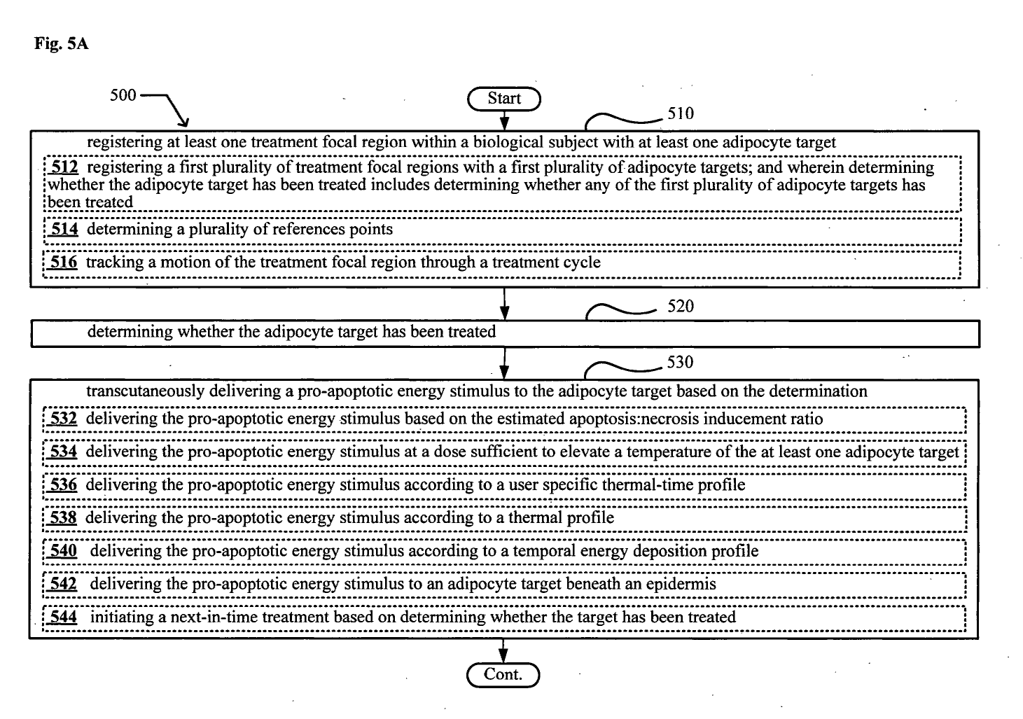

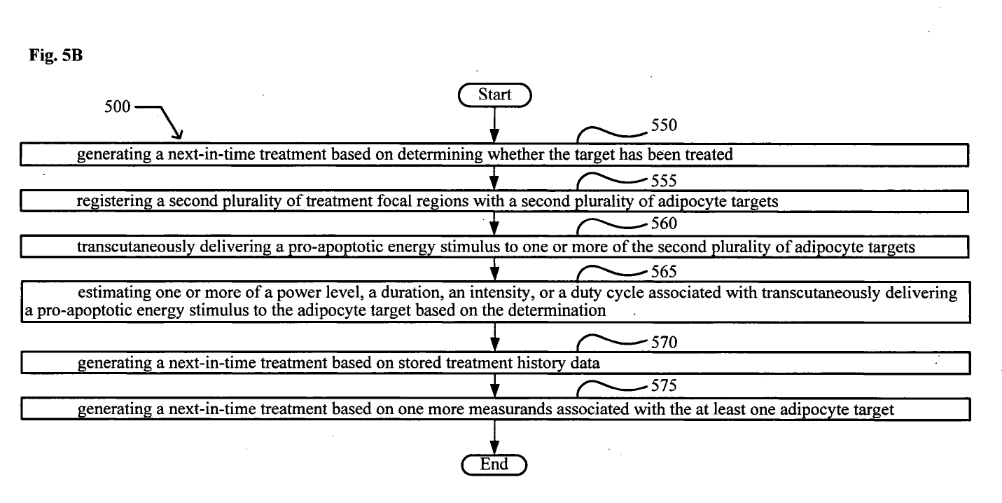

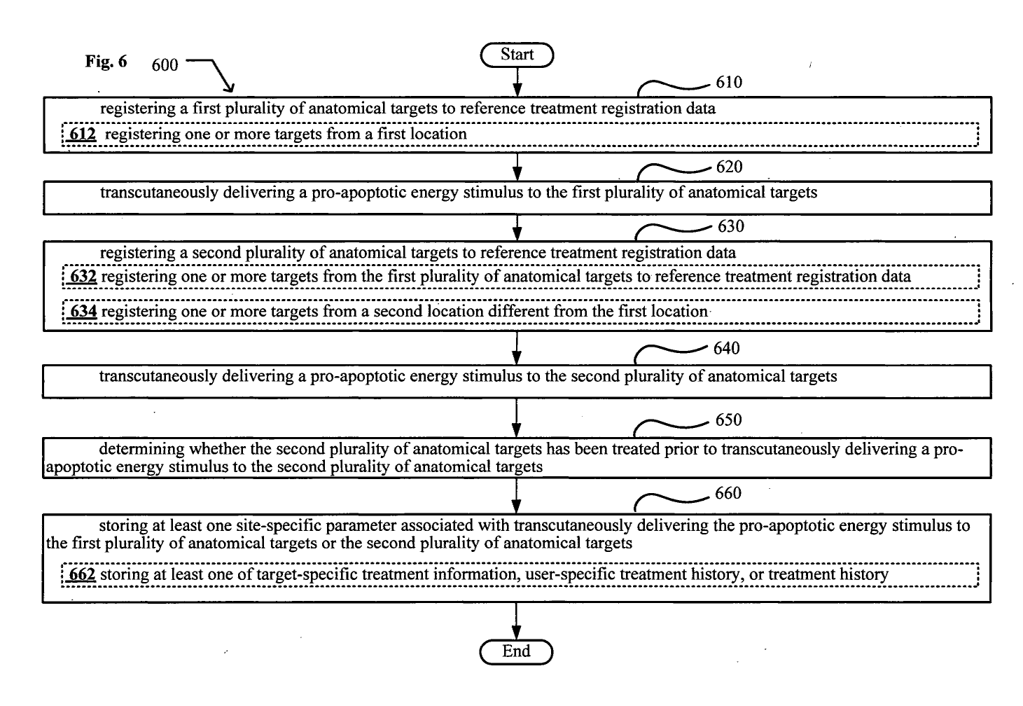

FIGS. 5 and 6 show a multi-pass transcutaneous energy delivery method 500. At 510, the method 500 includes registering at least one treatment focal region 104 within a biological subject with at least one adipocyte target. At 512, registering the at least one treatment focal regions 104 with the at least one adipocyte target includes registering a first plurality of treatment focal regions 104 with a first plurality of adipocyte targets. In an embodiment, registering the at least one treatment focal regions 104 with the at least one adipocyte target includes registering a first plurality of treatment focal regions with a first plurality of adipocyte targets; and wherein determining whether the adipocyte target has been treated includes determining whether any of the first plurality of adipocyte targets has been treated.

At 514, registering the at least one treatment focal region 104 with the at least one adipocyte target includes determining a plurality of references points. At 516, registering the at least one treatment focal region 104 with the at least one adipocyte target includes tracking a motion of the treatment focal region 104 through a treatment cycle. At 520, the method 500 includes determining whether the adipocyte target has been treated. In an embodiment, determining whether the adipocyte target has been treated includes determining whether any of the first plurality of adipocyte targets has been treated.

At 530, the method 500 includes transcutaneously delivering a pro-apoptotic energy stimulus 103 to the adipocyte target based on the determination. At 532, transcutaneously delivering the pro-apoptotic energy stimulus 103 includes delivering the pro-apoptotic energy stimulus 103 based on the estimated apoptosis:necrosis inducement ratio. At 534, transcutaneously delivering the pro-apoptotic energy stimulus 103 includes delivering the pro-apoptotic energy stimulus 103 at a dose sufficient to elevate a temperature of the at least one adipocyte target. In an embodiment, determining whether the adipocyte target has been treated includes classifying the at least one adipocyte target as treatment eligible or non-treatment eligible. In an embodiment, transcutaneously delivering the pro-apoptotic energy stimulus 103 includes delivering the pro-apoptotic energy stimulus 103 to a treatment eligible adipocyte target.

At 536, transcutaneously delivering the pro-apoptotic energy stimulus 103 includes delivering the pro-apoptotic energy stimulus 103 according to a user specific thermal-time profile. At 538, transcutaneously delivering the pro-apoptotic energy stimulus 103 includes delivering the pro-apoptotic energy stimulus 103 according to a thermal profile. At 540, transcutaneously delivering the pro-apoptotic energy stimulus 103 includes delivering the pro-apoptotic energy stimulus 103 according to a temporal energy deposition profile. At 542, transcutaneously delivering the pro-apoptotic energy stimulus 103 includes delivering the pro-apoptotic energy stimulus 103 to an adipocyte target beneath an epidermis. At 544, transcutaneously delivering the pro-apoptotic energy stimulus 103 includes initiating a next-in-time treatment based on determining whether the target has been treated. In an embodiment, transcutaneously delivering the pro-apoptotic energy stimulus 103 includes initiating a next-in-time treatment protocol based on determining whether the target has been treated. In an embodiment, transcutaneously delivering the pro-apoptotic energy stimulus 103 includes activating a treatment protocol based on a determination indicating that the adipocyte target has not been treated. In an embodiment, transcutaneously delivering the pro-apoptotic energy stimulus 103 includes deactivating a treatment protocol based on a determination indicating that the adipocyte target has been treated.

At 550, the method 500 includes generating a next-in-time treatment based on determining whether the target has been treated. At 555, the method 500 includes registering a second plurality of treatment focal regions 104 with a second plurality of adipocyte targets. At 560, the method 500 includes transcutaneously delivering a pro-apoptotic energy stimulus 103 to one or more of the second plurality of adipocyte targets. In an embodiment, generating a next-in-time treatment includes determining one or more of a power level, a duration, an intensity, or a duty cycle associated with transcutaneously delivering the pro-apoptotic energy stimulus 103 to one or more of the second plurality of adipocyte targets. At 565, the method 500 includes estimating one or more of a power level, a duration, an intensity, or a duty cycle associated with transcutaneously delivering a pro-apoptotic energy stimulus 103 to the adipocyte target based on the determination. At 570, the method 500 includes generating a next-in-time treatment based on stored treatment history data. At 575, the method 500 includes generating a next-in-time treatment based on one more measurands associated with the at least one adipocyte target.

FIG. 6 shows a multi-pass transcutaneous energy delivery method 600. At 610, the method 600 includes registering a first plurality of anatomical targets to reference treatment registration data. At 612, registering the first plurality of anatomical targets includes registering one or more targets from a first location. At 620, the method 600 includes transcutaneously delivering a pro-apoptotic energy stimulus 103 to the first plurality of anatomical targets. At 630, the method 600 includes registering a second plurality of anatomical targets to reference treatment registration data. At 632, registering the second plurality of anatomical targets includes registering one or more targets from the first plurality of anatomical targets to reference treatment registration data. At 634, registering the second plurality of anatomical targets includes registering one or more targets from a second location different from the first location. At 640, the method 600 includes transcutaneously delivering a pro-apoptotic energy stimulus 103 to the second plurality of anatomical targets. At 650, the method 600 includes determining whether the second plurality of anatomical targets has been treated prior to transcutaneously delivering a pro-apoptotic energy stimulus 103 to the second plurality of anatomical targets. At 660, the method 600 includes storing at least one site-specific parameter associated with transcutaneously delivering the pro-apoptotic energy stimulus 103 to the first plurality of anatomical targets or the second plurality of anatomical targets. At 662, storing the at least one site-specific parameter includes storing at least one of target-specific treatment information, user-specific treatment history, or treatment history.

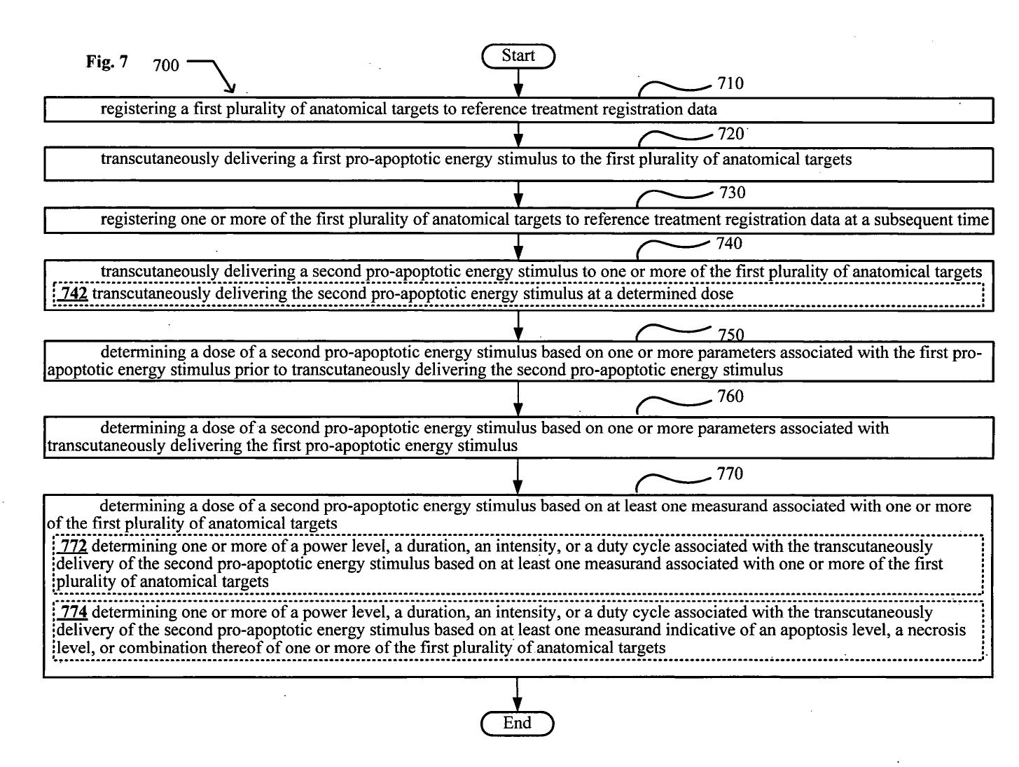

FIG. 7 shows a multi-pass transcutaneous energy delivery method 700.

At 710, the method 700 includes registering a first plurality of anatomical targets to reference treatment registration data. At 720, the method 700 includes transcutaneously delivering a first pro-apoptotic energy stimulus to the first plurality of anatomical targets. At 730, the method 700 includes registering one or more of the first plurality of anatomical targets to reference treatment registration data at a subsequent time. At 740, the method 700 includes transcutaneously delivering a second pro-apoptotic energy stimulus to one or more of the first plurality of anatomical targets. At 742, transcutaneously delivering the second pro-apoptotic energy stimulus to the one or more of the first plurality of anatomical targets includes transcutaneously delivering the second pro-apoptotic energy stimulus at a determined dose.

At 750, the method 700 includes determining a dose of a second pro-apoptotic energy stimulus based on one or more parameters associated with the first pro-apoptotic energy stimulus prior to transcutaneously delivering the second pro-apoptotic energy stimulus. At 760, the method 700 includes determining a dose of a second pro-apoptotic energy stimulus based on one or more parameters associated with transcutaneously delivering the first pro-apoptotic energy stimulus. At 770, the method 700 includes determining a dose of a second pro-apoptotic energy stimulus based on at least one measurand associated with one or more of the first plurality of anatomical targets. At 772, determining the dose of a second pro-apoptotic energy stimulus includes determining one or more of a power level, a duration, an intensity, or a duty cycle associated with the transcutaneous delivery of the second pro-apoptotic energy stimulus based on at least one measurand associated with one or more of the first plurality of anatomical targets. At 774, determining the dose of a second pro-apoptotic energy stimulus includes determining one or more of a power level, a duration, an intensity, or a duty cycle associated with the transcutaneous delivery of the second pro-apoptotic energy stimulus based on at least one measurand indicative of an apoptosis level, a necrosis level, or combination thereof of one or more of the first plurality of anatomical targets.

At least a portion of the devices and/or processes described herein can be integrated into a data processing system. A data processing system generally includes one or more of a system unit housing, a video display device, memory 134 such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for detecting position and/or velocity, control motors for moving and/or adjusting components and/or quantities). A data processing system can be implemented utilizing suitable commercially available components, such as those typically found in data computing/communication and/or network computing/communication systems.

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware in one or more machines or articles of manufacture), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation that is implemented in one or more machines or articles of manufacture; or, yet again alternatively, the implementer may opt for some combination of hardware, software, and/or firmware in one or more machines or articles of manufacture. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware in one or more machines or articles of manufacture.

The herein described subject matter sometimes illustrates different components contained within, or connected with, different other components. It is to be understood that such depicted architectures are merely examples, and that in fact, many other architectures can be implemented that achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably coupleable,” to each other to achieve the desired functionality. Specific examples of operably coupleable include, but are not limited to, physically mateable and/or physically interacting components, and/or wirelessly interactable, and/or wirelessly interacting components, and/or logically interacting, and/or logically interactable components.

In an embodiment, one or more components may be referred to herein as “configured to,” “configurable to,” “operable/operative to,” “adapted/adaptable,” “able to,” “conformable/conformed to,” etc. Such terms (e.g., “configured to”) can generally encompass active-state components and/or inactive-state components and/or standby-state components, unless context requires otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by the reader that each function and/or operation within such block diagrams, flowcharts, or examples can be implemented, individually and/or collectively, by a wide range of hardware, software, firmware in one or more machines or articles of manufacture, or virtually any combination thereof. Further, the use of “Start,” “End,” or “Stop” blocks in the block diagrams is not intended to indicate a limitation on the beginning or end of any functions in the diagram. Such flowcharts or diagrams may be incorporated into other flowcharts or diagrams where additional functions are performed before or after the functions shown in the diagrams of this application. In an embodiment, several portions of the subject matter described herein is implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal-bearing medium used to actually carry out the distribution. Non-limiting examples of a signal-bearing medium include the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

While particular aspects of the present subject matter described herein have been shown and described, it will be apparent to the reader that, based upon the teachings herein, changes and modifications can be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Further, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations, ” without other modifiers, typically means at least two recitations, or two or more recitations). Furthermore, in those instances where a convention analogous to “at least one of A, B, and C, etc.” is used, in general such a construction is intended in the sense of the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense of the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, the operations recited therein generally may be performed in any order. Also, although various operational flows are presented in a sequence(s), it should be understood that the various operations may be performed in orders other than those that are illustrated, or may be performed concurrently. Examples of such alternate orderings includes overlapping, interleaved, interrupted, reordered, incremental, preparatory, supplemental, simultaneous, reverse, or other variant orderings, unless context dictates otherwise. Furthermore, terms like “responsive to,” “related to, ” or other past-tense adjectives are generally not intended to exclude such variants, unless context dictates otherwise.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

1.-75. (canceled)

76. A transcutaneous energy delivery apparatus, comprising:
a real-time registration module configured to register one or more treatment focal regions with at least one anatomical target and to generate treatment registration information; and
an apoptosis induction module configured to transcutaneously deliver a pro-apoptotic energy stimulus to the one or more treatment focal regions based on the treatment registration information.

77. (canceled)

78. (canceled)

79. The transcutaneous energy delivery apparatus of claim 76, wherein the real-time registration module includes a computing device and a sensor component configured to detect a biological structure, the computing device configured to actuate an alignment of the one or more treatment focal regions with the at least one anatomical target based on a detected measurand associated with the biological structure.

80.-85. (canceled)

86. The transcutaneous energy delivery apparatus of claim 76, wherein the real-time registration module is configured to motion-track the movement of the at least one anatomical target, and real-time position the one or more treatment focal regions onto the at least one anatomical target.

87. The transcutaneous energy delivery apparatus of claim 76, wherein the real-time registration module includes a sensor component configured to determine a location of the at least one anatomical target by monitoring a metabolic process.

88.-90. (canceled)

91. The transcutaneous energy delivery apparatus of claim 76, wherein the real-time registration module is configured to register the one or more treatment focal regions with the anatomical target located using at least one of an artificial body surface marking, a tattoo, or a plurality of nanoparticle fiducial markers; and to generate treatment registration information.

92.-95. (canceled)

96. The transcutaneous energy delivery apparatus of claim 76, wherein the real-time registration module includes one or more computing devices configured to compare a detected dielectric profile associated with one or more subsurface anatomical targets to reference dielectric information and to generate treatment registration information based on the comparison.

97. The transcutaneous energy delivery apparatus of claim 76, wherein the real-time registration module includes
at least one sensor component configured to acquire a thermograph of the at least one anatomical target at one or more fields of view, and
one or more computing devices configured to determine a spatial frequency spectrum of at least a first subset of pixels of the thermograph, the one or more computing devices configured to generate treatment registration information based on a comparison of the spatial frequency spectrum of the first subset of pixels to reference thermograph information.

98. The transcutaneous energy delivery apparatus of claim 76, wherein the real-time registration module includes a sensor component configured to detect a location of a peripheral vascular bed, the real-time registration module configured to register the one or more treatment focal regions with the at least one anatomical target based on the detected location of the peripheral vascular bed.

99. (canceled)

100. (canceled)

101. The transcutaneous energy delivery apparatus of claim 76, wherein the real-time registration module is configured to identify groups of pixels in a thermograph indicative of at least one anatomical target imaged in the thermograph, and to generate treatment registration information representative of a parameter associated with a location and a dimension of the one or more treatment focal regions.

102.-105. (canceled)

106. The transcutaneous energy delivery apparatus of claim 76, wherein the real-time registration module includes a sensor component is configured to estimate an amount of adipose cells within the at least one anatomical target based on an interrogation energy stimulus.

107. The transcutaneous energy delivery apparatus of claim 76, wherein the apoptosis induction module includes one or more energy emitters configured to transcutaneously deliver a multi-focal pro-apoptotic energy stimulus.

108. (canceled)

109. The transcutaneous energy delivery apparatus of claim 76, wherein the apoptosis induction module includes one or more energy emitters configured to transcutaneously deliver the pro-apoptotic energy stimulus having frequency, power, duration, intensity, duty cycle, or pulse rate sufficient to induce apoptosis in a target amount of adipocytes.

110.-113. (canceled)

114. The transcutaneous energy delivery apparatus of claim 76, wherein the apoptosis induction module includes one or more energy emitters configured to transcutaneously deliver the pro-apoptotic energy stimulus at a dose sufficient to induce programmed cell death of adipocytes within the one or more treatment focal regions, without substantially inducing programmed cell death of overlying tissue.

115. (canceled)

116. The transcutaneous energy delivery apparatus of 76, wherein the apoptosis induction module includes one or more energy emitters that concurrently or sequentially deliver one or more of a pro-apoptotic electromagnetic energy stimulus, a pro-apoptotic electrical energy stimulus, a pro-apoptotic acoustic energy stimulus, or a pro-apoptotic thermal energy stimulus.

117.-130. (canceled)

131. The transcutaneous energy delivery apparatus of claim 76, wherein the apoptosis induction module is further configured to generate and store a next-in-time treatment protocol based on a comparison of the treatment registration information to patient specific treatment registration information.

132.-135. (canceled)

136. The transcutaneous energy delivery apparatus of claim 76, further comprising:
at least one of a transceiver or a receiver configured to receive an instruction to initiate transcutaneous delivery of the pro-apoptotic energy stimulus to the one or more treatment focal regions.

137. (canceled)

138. The transcutaneous energy delivery apparatus of claim 76, further comprising:
at least one of a transceiver or a transmitter configured to report a user status change in response to the transcutaneous delivery of the pro-apoptotic energy stimulus.

139. The transcutaneous energy delivery apparatus of claim 76, further comprising:
a physical coupling element configured to removably-attach the transcutaneous energy delivery apparatus to a biological surface of a biological subject.

140.-142. (canceled)

143. A transcutaneous energy delivery apparatus, comprising:
a real-time registration module configured to register one or more treatment focal regions with at least one anatomical target and to generate treatment registration information; and
an apoptosis induction module having
a first configuration operable to transcutaneously deliver a pro-apoptotic energy stimulus to the one or more treatment focal regions when the treatment registration information indicates that the one or more treatment focal regions coincide with the at least one anatomical target, and
a second configuration operable to cease transcutaneous delivery of a pro-apoptotic energy stimulus to the one or more treatment focal regions when the treatment registration information indicates that the one or more treatment focal regions do not coincide with the at least one anatomical target.

144. (canceled)

145. (canceled)

146. The transcutaneous energy delivery apparatus of claim 143, wherein the real-time registration module is further configured to provide treatment positioning information, via one or more of a visual, audio, haptic, or a tactile representation, associated with operation or placement of the transcutaneous energy delivery apparatus.

147. A method, comprising:
generating first-in-time treatment registration information indicative of an alignment of at least one treatment focal region with a first adipose depot target; and
transcutaneously delivering a pro-apoptotic energy stimulus to the at least one treatment focal region based on the first-in-time treatment registration information.

148. The method of claim 147, further comprising
storing target-specific treatment history associated with transcutaneously delivering the pro-apoptotic energy.

149.-151. (canceled)

152. The method of claim 147, further comprising:
reporting target registration information.

153.-155. (canceled)

156. The method of claim 147, further comprising:
reporting treatment protocol information.

157. (canceled)

158. (canceled)

159. The method of claim 147, further comprising:
determining a level of necrosis of the one or more adipose depot targets caused by transcutaneously delivering the pro-apoptotic energy stimulus.

160. The method of claim 147, further comprising:
detecting at least one tissue characteristic associated with the first adipose depot target at a plurality of sequential time points.

161. (canceled)

162. The method of claim 147, further comprising:
detecting a temperature profile of the first adipose depot target at a plurality of sequential time points.

163. The method of claim 147, further comprising
generating next-in-time treatment registration information indicative of an alignment of at least one treatment focal region with a subsequent adipose depot target.

164. The method of claim 147, further comprising
generating next-in-time treatment registration information indicative of an alignment of at least one treatment focal region with a subsequent adipose depot target based on a previous-in-time treatment data.

165. The method of claim 147, further comprising
updating a target-specific treatment history based on transcutaneously delivering the pro-apoptotic energy stimulus; and
generating next-in-time treatment registration information.

166.-170. (canceled)

171. The method of claim 147, wherein generating the first-in-time treatment registration information includes registering the at least one treatment focal region with an adipose depot located using at least one of an artificial body surface marking, a tattoo, or a plurality of nanoparticle fiducial markers.

172. (canceled)

173. (canceled)

174. The method of claim 147, wherein generating the first-in-time treatment registration information includes
determining a spatial frequency spectrum of at least a first subset of pixels of a thermograph associated with the first adipose depot target, and
generating the first-in-time treatment registration information by registering the at least one treatment focal region with the first adipose depot target based on a comparison of the spatial frequency spectrum of the first subset of pixels to reference thermograph information.

175.-188. (canceled)

189. The method of claim 147, further comprising:
generating second-in-time treatment registration information indicative of an alignment of at least one treatment focal region with the first adipose depot target or a second adipose depot target.

190. The method of claim 189, further comprising:
delivering a pro-apoptotic energy stimulus to the at least one treatment focal region based on the second-in-time treatment registration information.

191. The method of claim 147, further comprising:
comparing the first-in-time treatment registration information to a patient specific treatment protocol prior to transcutaneously delivering the pro-apoptotic energy stimulus.

192. The method of claim 147, further comprising:
determining treatment history of the first adipose depot target prior to transcutaneously delivering the pro-apoptotic energy stimulus.

193. The method of claim 147, further comprising:
determining whether to transcutaneously deliver the pro-apoptotic energy to the first adipose target by comparing the first-in-time treatment registration information to a user-specific treatment protocol; and
transcutaneously delivering the pro-apoptotic energy stimulus to the at least one treatment focal region based on the comparison of the first-in-time treatment registration information to the user-specific treatment protocol.

194. The method of claim 147, further comprising:
generating second-in-time treatment registration information indicative of an alignment of at least one treatment focal region with a second adipose depot target; and
transcutaneously delivering a pro-apoptotic energy stimulus to the at least one treatment focal region based on the second-in-time treatment registration information.

195. The method of claim 147, further comprising:
managing a duty cycle associated with transcutaneously delivering the pro-apoptotic energy stimulus based on a comparison of at least one real-time detected measurand associated with an adipose depot to reference adipose depot information.

196. The method of claim 147, further comprising:
managing a duty cycle associated with transcutaneously delivering the pro-apoptotic energy stimulus based on a comparison of a real-time detected adipose depot temperature to a reference temperature treatment protocol.

197. A transcutaneous energy delivery apparatus, comprising:
a target registration means for aligning a treatment focal region with an adipose depot target and for generating treatment protocol information; and
an apoptosis induction means for transcutaneously delivering an energy stimulus to the at least one treatment focal region.

198. The transcutaneous energy delivery apparatus of claim 197, further comprising:
a target tracking means including a sensor component and a computing device operably coupled to the sensor component and the apoptosis induction means, the target tracking means configured to register a treatment focal region location within the body of a biological subject relative to a reference location and to alter a duty cycle associated with the transcutaneous delivery of the energy stimulus based on the registering of the treatment focal region location within the body relative to the reference location.

199.-204. (canceled)
